(19) United States
(12) Patent Application Publication
(10) Pub. No.: US 2013/0156726 A1
(43) Pub. Date: Jun. 20, 2013

(54) ENDOMETRIAL STEM CELLS AND METHODS OF MAKING AND USING SAME

(71) Applicants: THOMAS E. ICHIM, San Diego, CA (US); XIAOLONG MENG, Witchita, KS (US); NEIL H. RIORDAN, Chandler, AZ (US)

(72) Inventors: THOMAS E. ICHIM, San Diego, CA (US); XIAOLONG MENG, Witchita, KS (US); NEIL H. RIORDAN, Chandler, AZ (US)

(73) Assignee: MediStem Laboratories, Inc., San Diego, CA (US)

(21) Appl. No.: 13/525,135

(22) Filed: Jun. 15, 2012

Related U.S. Application Data
(63) Continuation of application No. 12/127,697, filed on May 27, 2008, now abandoned.
(60) Provisional application No. 60/940,364, filed on May 25, 2007.
(60) Provisional application No. 60/987,880, filed on Nov. 14, 2007.

(51) Int. Cl. C12N 5/071; A61K 35/54; A61K 38/18; A61K 38/48; A61K 38/19

(52) U.S. Cl. CPC C12N 5/0682; A61K 38/4886; A61K 38/193; A61K 38/1858; A61K 38/1891; A61K 35/545
USPC 424/85.1; 435/366; 435/372; 424/93.7

(57) ABSTRACT

The invention provides pluripotent stem cells and methods for making and using pluripotent stem cells. Pluripotent stem cells, among other things, can differentiate into various cell lineages in vitro, ex vivo and in vivo. Pluripotent stem cells, among other things, can also be used to produce conditioned medium.

### RELATED APPLICATIONS

This application claims the benefit or priority of U.S. application Ser. No. 60/940,364, filed May 25, 2007, and U.S. application Ser. No. 60/987,880, filed Nov. 14, 2007, which are expressly incorporated herein by reference.

### INTRODUCTION

Stem cell therapy offers the possibility of treating many previously uncurable diseases. Numerous types of stem cells exist and there are efforts to identify additional stem cells. Broadly speaking, stem cells can be divided into embryonic and adult types. While embryonic stem cells possess great ability to proliferate, specific induction of their controlled differentiation has been elusive. The fear of embryonic stem cells causing teratomas has been a major obstacle to their clinical development. Embryonic stem cells are described in U.S. Pat. No. 5,843,780. Adult stem cells such as bone marrow, cord blood, adipose derived and amnionic fluid derived have demonstrated regenerative potential in a variety of diseases and degenerative disorders, however, these cell types are limited by: availability, invasiveness of extraction, and in some cases limited proliferative capacity. What is currently needed are stem cells that overcome these deficiencies, while not possessing the fear of karyotypic abnotinalities during culture and possibility of oncogenesis.

### SUMMARY

Disclosed are mammalian (e.g., human) cells, populations and pluralitires of cells and cell cultures that can be obtained or derived from menstrual tissue or blood that possess pluripotency (i.e., the ability to differentiate into various cell types). The mammalian pluripotent stem cells can be characterized by expression of particular phenotypic markers (e.g., CD29, CD41a, CD90, etc.), or lack of expression of particular phenotypic markers (e.g., NeuN, CD9, CD62, CD59, etc.), a relatively rapid rate of cellular division (e.g., a doubling rate of between about once every 12-24 or 24-48 hours), adherent growth in tissue culture, and maintenance of phenotypic and karyotypic integrity after extended number of cell divisions (doublings).

The invention provides mammalian (e.g., human) pluripotent stem cells. In one embodiment, a pluripotent stem cell expresses a marker selected from CD29, CD41 a, CD44, CD90, and CD105, and has an ability to proliferate at a rate of 0.5-1.5 doublings per 24 hours in a growth medium. In another embodiment, a pluripotent stem cell expresses a marker selected from NeuN, CD9, CD62, CD59, Actin, GFAP, NSE, Nestin, CD73, SSEA-4, hTERT, Oct-4, and tubulin. In additional embodiments, a pluripotent stem cell expresses a marker selected from hTERT and Oct-4, but does not express a STRO-1 marker, and has an ability to undergo cell division in less than 24 hours in a growth medium. In further embodiments, pluripotent stem cell expresses a STRO-1 marker, and has an ability to proliferate at a rate of 0.5-0.9 doublings per approximately 24 hours (e.g., 20-24) in a growth medium. In still further embodiments, a pluripotent stem cell does not express one or more of CD34, alpha myosin, insulin or albumin markers, or does not detectably stain with the adipocyte-labeling dye AdipoRed or the osteogenic-specific dye Alizarin Red (e.g., as determined by immunohistochemistry). In yet additional embodiments, a pluripotent stem cell expresses a mesenchymal cell marker (e.g., CD54, CD106, an HLA-I marker, vimentin, ASMA, collagen-1, or fibronectin, but not a HLA-DR, CD1 17, or a hemopoietic cell marker).

In particular aspects, a pluripotent stem cell expresses or produces matrix metalloprotease 3 (MMP3), matrix metalloprotease 10 (MMP10), GM-CSF, PDGF-BB or angiogenic factor ANG-2. In an additional particular aspect, a pluripotent stem cell expresses an elongated fibroblast-like morphology. In a further particular aspect, a pluripotent stem cell has an adherent property (e.g., adheres to a substrate in a culture).

A mammalian (e.g., human) pluripotent stem cell can be derived from or can originate from endometrium, endometrial stroma, endometrial membrane, or menstrual blood. A mammalian (e.g., human) pluripotent stem cell need not be derived from or originate from a cell that was derived or originated from endometrium, endometrial stroma, endometrial membrane, or menstrual blood. For example, a mammalian (e.g., human) pluripotent stem cell can be a progeny of a cell that was derived or originated from endometrium, endometrial stroma, endometrial membrane, or menstrual blood. Thus, mammalian (e.g., human) pluripotent stem cells include progeny cells (e.g., clonal pluripotent stem cells or differentiated froms) not derived or obtained from endometrium, endometrial stroma, endometrial membrane, or menstrual blood.

Mammalian (e.g., human) pluripotent stem cells include cells transfected with a nucleic acid. Such nucleic acids can encode proteins for expression in vitro, ex vivo or in vivo.

Mammalian (e.g., human) pluripotent stem cells are capable of, among other things, differentiating into particular cell lineages. In particular embodiments, pluripotent stem cells are capable of differentiating into adipogenic, endothelial, hepatic, osteogenic, neural, pancreatic or myocytic cell lineage. In additional particular embodiments, pluripotent stem cells are capable of differentiating into cells of a pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, or mesentery tissue.

Mammalian (e.g., human) pluripotent stem cells include cells which have a stable karyotype for one or more cell divisions. In particular embodiments, a pluripotent stem cell has a stable karyotype for at least 10, 20, 30, 40, 50, 60, 70, 80, 90, 100 or more cell divisions.

Mammalian (e.g., human) pluripotent stem cells include cells which do not readily undergo transformation. In particular embodiments, a pluripotent stem cell is expanded 2 fold, 5 fold, 10 fold, 20 fold, 30 fold, 40 fold, 50 fold, 60 fold, 70 fold or more without cell transformation in vitro or in vivo.

Mammalian (e.g., human) pluripotent stem cells include cells capable of stimulating, inducing, increasing, promoting, enhancing or augmenting a reparative process in a host. Mammalian (e.g., human) pluripotent stem cells also include cells capable of suppressing, inhibiting, reducing, decreasing, preventing, blocking, limiting or controlling a T cell mediated response in vitro or in vivo. Mammalian (e.g., human) pluripotent stem cells additionally include cells capable of stimulating angiogenesis, inhibiting fibrosis or scar tissue formation, inhibiting inflammation, inhibiting undesired or pathological apoptosis (after heart attack, a stroke, or liver failure, cells started to undergo programmed cell death in a pathological manner, or differentiating). Mammalian (e.g., human) pluripotent stem cells further include cells capable of stimulating endogenous progenitor cell proliferation (i.e., a subject's progenitor cells), stimulation of endogenous stem cell proliferation (i.e., a subject's stem cells), stimulation of endogenous progenitor cell differentiation, stimulation of endogenous stem cell differentiation, stimulation of exogenous progenitor cell proliferation, stimulation of exogenous stem cell proliferation, stimulation of exogenous progenitor cell differentiation, stimulation of exogenous stem cell differentiation.

Mammalian (e.g., human) pluripotent stem cells include isolated or purified cells (including progeny and cells differentiated therefrom). Mammalian (e.g., human) pluripotent stem cells include population and pluralities of such cells, as well as cultures of such cells (including progeny and cells differentiated therefrom). Relative proportions of pluripotent stem cells can vary. Non-limiting embodiments include pluripotent stem cells that are at least 25%, 50%, 75%, 90% or more of the population, plurality or culture of cells; 75%, 80%, 85%, 90%, 95% or more of said cells of the population, plurality or culture express a marker selected from CD49C, CD105, CD44, CD90, and OCT4; or 20%, 15%, 10%, 5% or less of said cells of the population, plurality or culture express a marker selected from CD34, CD45 and CD 133. In such collections of cells, the pluripotent stem cells can proliferate or increase in numbers with less than 50%, 40%, 30%, 25%, 20%, 15%, 10%, 5% or less of the cells differentiating. Such collections of cells can include cells differentiated from pluripotent stem cell

Mammalian (e.g., human) pluripotent stem cells include co-cultures of cells. In one embodiment, a co-culture includes a human pluripotent stem cell (or population or plurality of cells) and one or more second cells. In particular aspects, the second cells can be T cells, dendritic cells, NK cells, monocytes, macrophages PBMCs, or stem cells (adult or embryonic, totipotent, pluripotent, multipotent, a progenitor or a differentiated cell. Such co-cultures can be used to induce, stimulate, promote, increase or augment proliferation or differentiation of the second cells.

The invention also provides culture medium incubated with mammalian (e.g., human) pluripotent stem cells for a period of time, which can be referred to as conditioned medium. In particular embodiments, the culture medium is incubated with mammalian (e.g., human) pluripotent stem cells for about 1-72 hours, 3-7 days, or more.

Conditioned medium can include factors produced or secreted by pluripotent stem cells, such as matrix metalloprotease 3 (MMP3), matrix metalloprotease 10 (MMP10), GM-CSF, PDGF-BB or angiogenic factor ANG-2. Such medium has various activities, including the ability to, among other things, stimulate, increase, induce, enhance or augment cell survival, viability, growth, proliferation or differentiation of a cell, such as a totipotent stem or a human umbilical vein endothelial cell; stimulate, increase, induce, enhance or augment hematopoiesis; inhibit, reduce, decrease, prevent, block control or limit inflammation.

Conditioned medium can be manipulated, such as separated from cells (e.g., aspirating or dispensing in a vessel or container), harvested, concentrated, lyophilized, etc.

The invention further provides methods of treating a subject with mammalian (e.g., human) pluripotent stem cells, or conditioned medium. Exemplary non-limiting conditions to be treated, subjects to be treated and objectives of treatment include: ischemia in a tissue or organ (e.g., cardiac or pulmonary tissue, limb, or kidney); stroke, pulmonary fibrosis, or diabetic limb; fibrosis or scar tissue formation (e.g., in a tissue or organ, such as cardiac or pulmonary tissue, limb, liver, pancreas, or kidney); to increase or improve a pancreas or liver function (e.g., increase numbers or proliferation of islet cells, numbers or proliferation of hepatocytes, or insulin); diabetes, liver failure, cirhossis, liver or pancreas fibrosis, or hepatitis; to increase osteocyte numbers, osteocyte formation or an osteocyte function; a bone fracture or break, or is in need of a prosthesis in a joint; increasing or improving pulmonary or cardiac function; cardiac disease, artherosclerosis, myocardial infarction (Heart Attack), cardiac infection, heart failure, ischemic heart failure, high blood pressure (Hypertension), or pulmonary hypertension, idiopathic pulmonary fibrosis, stroke, congenital heart disease (CHD), congestive heart failure, angina, myocarditis, coronary artery disease, cardiomyopathy, dilated cardiomyopathy, hypertrophic cardiomyopathy, endocarditis, diastolic dysfunction, cerebrovascular disease, valve disease, mitral valve prolapse, venous thromboembolism or arrhythmia; a neurological or muscular disease or disorder (e.g., multiple sclerosis (MS), spinal cord injury, muscular dystrophy (Becker's or Duchenne's), amyotrophic lateral sclerosis (ALS; Lou Gehrig's disease or classical motor neuron disease), autism, progressive bulbar palsy (progressive bulbar atrophy), pseudobulbar palsy, primary lateral sclerosis (PLS), progressive muscular atrophy, spinal muscular atrophy (SMA, including SMA type I—Werdnig-Hoffmann disease, SMA type II, or SMA type III—Kugelberg-Welander disease), Fazio-Londe disease, Kennedy disease (progressive spinobulbar muscular atrophy), congenital SMA with arthrogryposis, or post-polio syndrome (PPS); inhibition of inflammation, inhibition of undesirable or pathological apoptosis; any subject that would benefit from a stem cell or conditioned medium therapy (e.g., new cells or new tissue, stimulation of endogenous progenitor cell proliferation, stimulation of endogenous stem cell proliferation, stimulation of endogenous progenitor cell differentiation, or stimulation of endogenous stem cell differentiation; increased numbers or improved function, healing or repair of adipogenic, endothelial, hepatic, osteogenic, pancreatic, neural or myocytic cells, comprising administering adipogenic, endothelial, hepatic, osteogenic, pancreatic, neural or myocytic cells to the subject in an amount sufficient to provide increased numbers or improved function, healing or repair of adipogenic, endothelial, hepatic, osteogenic, pancreatic, neural or myocytic cells; diabetes, liver failure, a neurological dirorder or disease, or lung fibrosis; increase T regulatory cells; to treat melanoma; to treat an autoimmune disorder; immunological rejection of a transplant, transplant fibrosis or graft failure; stimulate hematopoiesis; and stimulate angiogenesis.

The invention further provides isolated or purified undifferentiated cells obtained from endometrium, endometrial stroma, endometrial membrane, or menstrual blood. In one embodiment, a cell hasa fibroblast-like morphology and has an ability to differentiate into oen or more different cell types. In particlaur aspects, the undifferentiated cells can differentiate into a cell of a pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, or mesentery tissue.

The invention moreover provides progeny cells of mammalian (e.g., human) pluripotent stem cells. In one embodiment, a progeny is a progenitor or precursor cell of an adipogenic, endothelial, hepatic, osteogenic, neural, pancreatic or myocytic cell into which a pluripotent stem cell differentiates. In another embodiment, a progeny is a developmental intermediate of a cell into which a mammalian (e.g., human) pluripotent stem cell differentiates (e.g., an adipogenic, endothelial, hepatic, osteogenic, neural, pancreatic or myocytic cell). Such progeny cells can be characterized as expressing particular markers, not detectably expressing particular markers, having a defined doubling time or mmorphology, or other characteristics as set froth herein.

### DRAWING DESCRIPTIONS

FIG. 1 shows representative morphology of the Menstrual Blood Derived Reparative Cells After Overnight Culture (100×).

FIG. 2 shows representative morphology of Menstrual Membrane Derived Reparative Cells After Overnight Culture (100×).

FIG. 3 shows representative morphology of Menstrual Blood Derived Reparative Cells After 2 Week Culture (100×). Cells all assume a fibroblastoid-like morphology and were adherent to the tissue culture flask.

FIG. 4 shows representative morphology of Menstrual Membrane Derived Reparative Cells After 48 hour Culture (100×). Cells exhibited a similar morphology to cells derived from menstrual blood.

FIG. 5 shows a representative 96 well plate of cloning of Menstrual Blood Derived Reparative Cells and the doubling rate of cells plated at a 1 cell per well concentration (40×).

FIG. 6 shows phenotyping at early passage, and a phenotypic difference between cells extracted from more slowly proliferating cells (doubling about every 24-48 hours) compared to more highly proliferating cells (doubling within 24 hours, typically once every 20-24 hours).

FIG. 7 shows phenotyping at a later passage (40 doublings), and that the phenotypic differences between more slowly proliferating cells compared to more highly proliferating cells was maintained.

FIG. 8 shows phenotyping of highly proliferating cells by immunohistochemistry for the indicated markers.

FIG. 9 shows phenotyping of highly proliferating cells by immunohistochemistry for the indicated markers.

FIG. 10 shows the results of flow cytometric and microscopic analysis of a heterogeneous population of menstrual blood derived mononuclear cells as described in Example 6. A gradual decrease in percentage positivity of various cell markers associated with stem cells is observed with increased passages.

FIG. 11 shows that highly proliferating stem cells maintain karyotypic normality at 70-80 doublings.

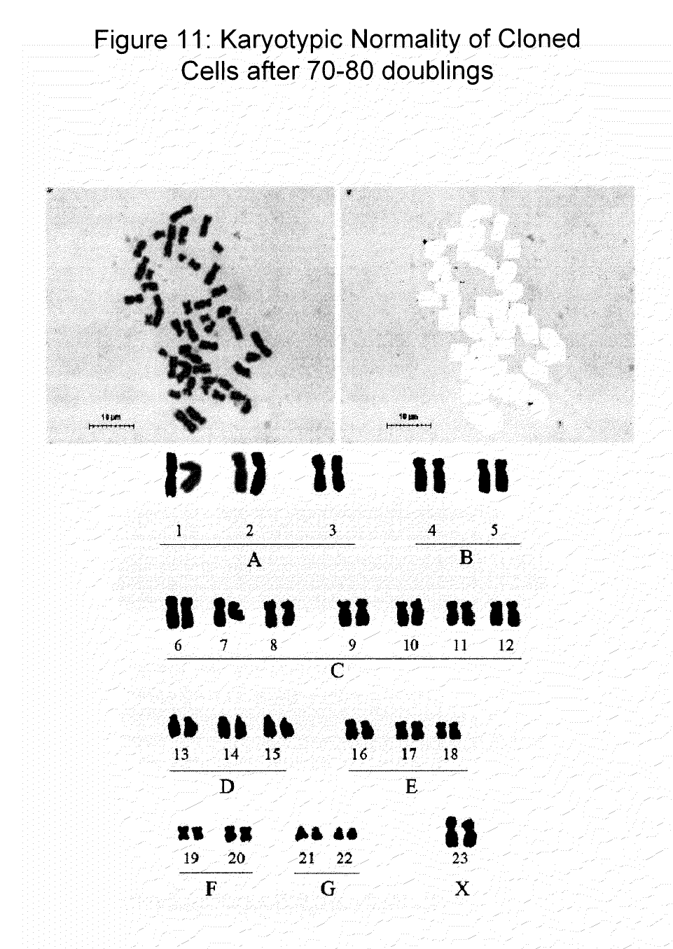

FIG. 12A-12M show that stem cells were capable of differentiating into a variety of different cell types, including cells with A) adipocyte-like morphology; B) an osteocyte-like morphology; C) myocyte (Alpha Actinin+); D) Skeletal myocyte (Skeletal Myosin+); E) endothelial cells (CD34+); F) endothelial cells (CD62+); G) hepatocyte-like morphology; H) hepatic-specific protein (albumin+): I) pancreatic-like cells (insulin producing); J) neural (GFAP+); K) neural (Nestin+); L) pulmonary epithelial; and M) cardiac differentiation (ProSP-C+).

FIG. 13 shows data indicating that the stem cells produced a substantially higher level of MMP-3 and 10, as well as GM-CSF, PDGF-BB, and Angiopoietin-2, as compared to control BioE cord blood derived mesenchymal stem cells.

FIG. 14 shows a dose dependent stimulation of bone marrow mononuclear cell proliferation in cultures treated with medium conditioned with pluripotent stem cells (ERC supernatant).

FIG. 15 shows a stimulation of human umbilical vein endothelial cell (HUVEC) proliferation in cultures treated with medium conditioned with pluripotent stem cells (ERC supernatant).

FIG. 16 shows a representative control and pluripotent stem cell treated (ERC) mouse limb ischemia animal model, indicating that treatment promoted angiogenesis in the ischemic limb.

FIG. 17 shows a lack of allostimulatory activity of pluripotent stem cells.

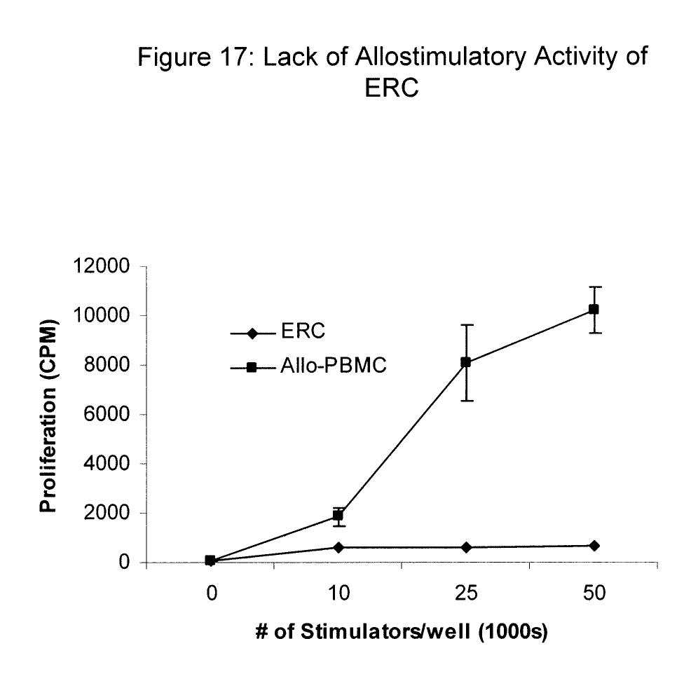

FIG. 18 shows that pluripotent stem cells actively suppress ongoing mixed lymphocyte reaction (MLR).

FIG. 19 shows that pluripotent stem cells suppress IFN-gamma production.

FIG. 20 shows that pluripotent stem cells stimulate IL-4 production.

FIG. 21 shows a that pluripotent stem cells suppress TNF-alpha production.

### DETAILED DESCRIPTION

The invention provides, among other things, mammalian (e.g., human) pluripotent stem cells, populations and pluralities of mammalian (e.g., human) pluripotent stem cells, and cultured populations and pluralities of mammalian (e.g., human) pluripotent stem cells. Such pluripotent stem cells are characterized by various features, including, for example, the presence or absence of various phenotypic markers, the ability to undergo cell division within a given time period in a suitable growth medium, the ability to produce certain proteins, and a characteristic morphology. In one embodiment, pluripotent stem cells express a marker selected from CD29, CD41a, CD44, CD90, and CD105. In another embodiment, pluripotent stem cells express a marker selected from NeuN, CD9, CD62, CD59, Actin, GFAP, NSE, Nestin, CD73, SSEA-4, hTERT, Oct-4, and tubulin. In a further embodiment, pluripotent stem cells do not express a marker selected from CD34, alpha myosin, insulin or albumin. In still another embodiment, a human pluripotent stem cell does not detectably stain with the adipocyte-labeling dye AdipoRed or the osteogenic-specific dye Alizarin Red.

Additional markers of pluripotent stem cells include markers that can be expressed by mesenchymal stem cells. Mesenchymal stem cells are pluripotent stem cell progenitor, such as a blast cell of one or more mesenchymal cell lineages, including bone, cartilage, muscle, fat tissue, bone marrow, marrow stroma, dermis and astrocytes. Mesenchymal stem cells can be found in, for example, blood and periosteum. Non-limiting examples of mesenchyme stem cell markers include one or more of: CD54, CD106, an HLA-I marker, vimentin, ASMA, collagen-1, or fibronectin, but not a HLA-DR, CD1 17, or a hemopoietic cell marker.

Pluripotent stem cells have ability to undergo cell division or proliferate at a relatively defined rate, which for convenience is referred to herein as “doublings” or a “doubling time” within a certain time period (a doubling refers to one round of cell division). In one embodiment, pluripotent stem cells proliferate at a rate of 0.5-1.5 doublings per 24 hours in a growth medium. In a particular aspect, a pluripotent stem cell that expresses a marker selected from hTERT and Oct-4, but does not express a STRO-1 marker has an ability to undergo cell division in less than 24 hours in a suitable growth medium. In another particular aspect, a pluripotent stem cell that expresses a STRO-1 marker has an ability to proliferate at a rate of 0.5-0.9 doublings per 24 hours in a growth medium. Such proliferation rates can be established in any suitable medium. Non-limiting exemplary cell medium are a liquid medium such as DMEM, alpha-MEM or RPMI. Other suitable medium for pluripotent stem cell maintenance, growth and proliferation would be known to the skilled artisan. Such media can include one or more of supplements, such as albumin, essential amino acids, non essential amino acids, L-glutamine, a thyroid hormone, vitamins, etc.

Pluripotent stem cells include cells that produce proteins, such as proteins that may have therapeutic value. In one embodiment, pluripotent stem cells produce a matrix metalloprotease 3 (MMP3), matrix metalloprotease 10 (MMP10), GM-CSF, PDGF-BB or angiogenic factor ANG-2.

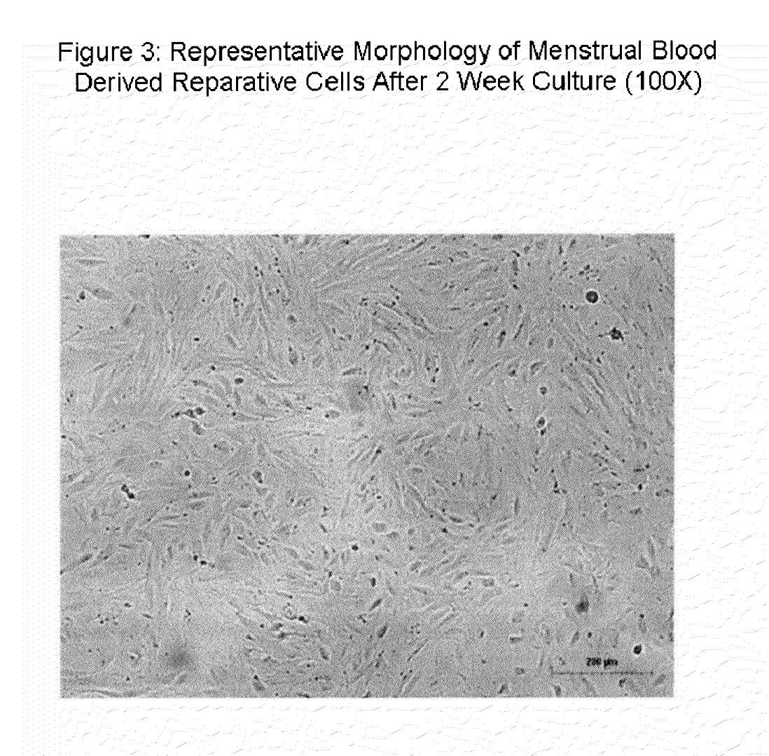

Pluripotent stem cells have a defined morphology. In one embodiment, a pluripotent stem cell has an elongated fibroblast-like morphology (FIG. 3, after 2 weeks of culture).

Pluripotent stem cells can also have additional features. For example, in one embodiment, a pluripotent stem cell has an adherent property. In a particular aspect, a pluripotent stem cell adheres to a substrate (e.g., polyvinyl chloride or other plastic, glass, fibers, gelatinous substrates, etc.). In an additional aspect, a pluripotent stem cell can form a monolayer on a substrate.

Pluripotent stem cells also include cells that have a stable karyotype over one or more doublings (cell divisions). In one embodiment, a pluripotent stem cell has a stable karyotype for at least 10, 20, 30, 40, 50, 60, 70, 80, 90, 100 or more cell divisions (doublings). In another embodiment, a pluripotent stem cell is capable of being expanded 2 fold, 5 fold, 10 fold, 20 fold, 30 fold, 40 fold, 50 fold, 60 fold, 70 fold or more without karyotypic variation.

In pluripotent stem cell populations, pluralities and cultures, there may be some percentage of pluripotent stem cells that exhibit karyotype variation. Such cells will typically represent a smaller proportion of pluripotent stem cells than the pluripotent stem cells that have a stable karyotype. In particular aspects, the relative proportion of pluripotent stem cells that have a stable karyotype will represent greater than about 60%, 70%, 80%, 90%-95% or more (e.g., 96%, 97%, 98%, etc. . . . 100%) of the total number of pluripotent stem cells present in the population, plurality or culture.

A “pluripotent stem cell” is a cell with the ability to self-renew (clonally proliferate) and remain undifferentiated. A stem cell is therefore not terminally differentiated and not at the end stage of a differentiation pathway. Under appropriate conditions or stimuli, to pluripotent stem cell can differentiate. Thus, when a stem cell divides, a daughter cell can either remain a stem cell or progress towards terminal differentiation.

Pluripotent stem cells further include cells that are capable of being expanded without oncogenic transformation. In one embodiment, a pluripotent stem cell has a stable karyotype for at least 10, 20, 30, 40, 50, 60, 70, 80, 90, 100 or more cell divisions (doublings) without cell transformation. In another embodiment, a pluripotent stem cell is capable of being expanded 2 fold, 5 fold, 10 fold, 20 fold, 30 fold, 40 fold, 50 fold, 60 fold, 70 fold or more without cell transformation.

The term “transformed” and grammatical variations thereof, when used in reference to a pluripotent stem cell, refers to oncogenic transformation, which can result in development of a tumor or cancer. A non-limiting in vitro method of determining whether cells are transformed (e.g., oncogenic transformation) include growth of a cell in a serum free medium. A non-limiting in vivo method of determining whether cells have become transformed is determined by the absence of tumors in nude mice. For example, evaluation of various organs and tissues of nude mice four months after injection with about 0.5 million of human pluripotent stem cells did not detect tumors.

Pluripotent stem cells additionally include cells that are capable of differentiating into various cell lineages, in vitro or in vivo. In one embodiment, a pluripotent stem cell is capable of differentiating into adipogenic, endothelial, hepatic, osteogenic, neural, pancreatic or myocytic cell lineage. In another embodiment, a pluripotent stem cell is capable of differentiating into cells of a pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, or mesentery tissue.

The invention therefore also provides cells differentiated with respect to mammalian (e.g., human) pluripotent stem cells, wherein the cells are progeny of a mammalian (e.g., human) pluripotent stem cell. In one embodiment, a cell is a progeny cell differentiated with respect to mammalian (e.g., human) pluripotent stem cell and is a developmental progenitor or precursor cell of an adipogenic, endothelial, hepatic, osteogenic, neural, pancreatic or myocytic cell. In another embodiment, a cell is a progeny cell that is a developmental intermediate with respect to mammalian (e.g., human) pluripotent stem cell and a adipogenic, endothelial, hepatic, osteogenic, neural, pancreatic or myocytic cell. In a further embodiment, a cell is a progeny cell differentiated with respect to mammalian (e.g., human) pluripotent stem cell and is a differentiated adipogenic, endothelial, hepatic, osteogenic, neural, pancreatic or myocytic cell.

A “progeny” of a pluripotent stem cell refers to any and all cells derived from pluripotent stem cells as a result of clonal proliferation or differentiation. As used herein, a “progenitor cell” is a parent cell committed to give rise to a distinct cell lineage by a series of cell divisions. Specific progenitor cell types may sometimes be identified by markers. A “precursor cell” refers to a cell from which another cell is formed. It encompasses a cell that precedes the existence of a later, more developmentally mature cell. In contrast to the maturation of progenitor cells, which is marked by cell division, the developmental maturation of a precursor cell may include any number of processes or events, including, but not limited to, differential gene expression, or change in size, morphology, or location. As used herein, both progenitor and precursor cells are progeny of and distinct from a pluripotent stem cell. A “developmental intermediate” cell refers to any cell that is either a progenitor or precursor cell that is distinct from the pluripotent stem cells and the ultimately differentiated cell type.

Pluripotent stem cells moreover include cells that are capable of modulating an immune cell or immune response, in vitro or in vivo. In one embodiment, a pluripotent stem cell is capable of suppressing, inhibiting, reducing, decreasing, preventing, blocking, limiting or controlling a T cell mediated response in vitro or in vivo. In particular aspects, a T cell mediated response comprises PBMC proliferation, production of a cytokine, production of interferon gamma, or production of TNF alpha. In further aspects, pluripotent stem cells are capable of suppressing mixed lymphocyte reactions, as well as give rise to T regulatory cells in co-cultures of pluripotent stem cells and PBMCs, with cells present in or derived from PBMCs (e.g., a CD4+ T cell or an NK T cell), or in vivo.

Pluripotent stem cells yet additionally include cells (or progeny) that are capable of stimulating, inducing, increasing, promoting, enhancing or augmenting a reparative process ex vivo or in vivo (e.g., in a subject or a host). A “reparative” or “regenerative” process refers to any activity that contributes to amelioration or improvement of damaged or diseased cells, tissues or organs. A reparative or regenerative process can be direct, for example, pluripotent stem cells differentiating into cells (progeny) that replace damaged or diseased cells (e.g., insulin producing islet cells) in a subject. The reparative or regenerative process can be indirect, for example, although not wishing to be bound by theory, pluripotent stem cells may secrete factors, such as those set forth herein (e.g., PDGF-BB, etc.) or others that elicit the subjects' endogenous stem cells or differentiated cells to become activated, to proliferate or to differentiate thereby repairing the damaged tissue or cells (e.g., insulin producing islet cells). Non-limiting reparative and regenerative activities include decreasing, or reducing, fibrosis, stimulating, increasing, inducing, enhancing or augmenting angiogenesis, and stimulating, increasing, inducing, enhancing or augmenting of vascular function. Thus, the ability of pluripotent stem cells (or progeny) to have any of the foregoing capabilities in vivo may only need to be transient and need not require short or long term viability in vivo.

Representative non-limiting examples of a reparative process include, for example, stimulating, inducing, increasing, promoting, enhancing or augmenting angiogenesis, reducing, decreasing, inhibiting, controlling, limiting, blocking or preventing fibrosis or scar tissue formation, reducing, decreasing, inhibiting, controlling, limiting, blocking or preventing inflammation, reducing, decreasing, inhibiting, controlling, limiting, blocking or preventing undesired or pathological apoptosis (e.g., after heart attack, a stroke, or liver failure, cells start to undergo programmed cell death in a pathological manner). Additional representative examples of a reparative process include, for example, stimulating, inducing, increasing, promoting, enhancing or augmenting endogenous progenitor cell proliferation, stimulating, inducing, increasing, promoting, enhancing or augmenting endogenous stem cell proliferation, stimulating, inducing, increasing, promoting, enhancing or augmenting endogenous progenitor cell differentiation, stimulating, inducing, increasing, promoting, enhancing or augmenting endogenous stem cell differentiation, stimulating, inducing, increasing, promoting, enhancing or augmenting exogenous progenitor cell proliferation, stimulating, inducing, increasing, promoting, enhancing or augmenting exogenous stem cell proliferation, stimulating, inducing, increasing, promoting, enhancing or augmenting exogenous progenitor cell differentiation and stimulating, inducing, increasing, promoting, enhancing or augmenting exogenous stem cell differentiation. Thus, pluripotent stem cells (or progeny) can be used in treatment and therapeutic methods to effect treatment of a subject.

Pluripotent stem cells of the invention include pluripotent stem cell populations and pluralities of pluripotent stem cells (progeny thereof), and cultures of pluripotent stem cells (cell cultures, and progeny cultures). A population or plurality or culture of pluripotent stem cells (or progeny) mean that there are a collection of such cells. In various embodiments, a pluripotent stem cell population, plurality of pluripotent stem cells or culture of pluripotent stem cells (or progeny) include mammalian (e.g., human) pluripotent stem cells that represent at least 25%, 50%, 75%, 90% or more of the total number of cells in the population or plurality or culture. Such cell populations and pluralities are considered enriched for pluripotent stem cells (e.g., cells that express a marker such as CD29, CD41a, CD44, CD90, CD105, hTERT Oct-4, NeuN, CD9, CD62, CD59, actin, etc., or do not express a marker such asCD34, alpha myosin, insulin, albumin, etc.).

In a population or plurality of pluripotent stem cells, or in a culture of pluripotent stem cells, a majority of cells, but not all cells present may or may not express a particular phenotypic marker indicative of a pluripotent stem cell. Such cells are typically present in the population, plurality or culture at a smaller percentage of the total number of pluripotent stem cells present. In various embodiments, a pluripotent stem cell population, plurality of pluripotent stem cells or culture of pluripotent stem cells include cells in which greater than about 50%, 60%, 70%, 80%, 90%-95% or more (e.g., 96%, 97%, 98%, etc. . . . 100%) of the cells express a particular phenotypic marker. In particular aspects, 75%, 80%, 85%, 90%, 95% or more of the population, plurality of pluripotent stem cells or culture of pluripotent stem cells express a marker selected from CD29, CD41a, CD44, CD 105, CD90, and OCT4. In various embodiments, a pluripotent stem cell population, plurality of pluripotent stem cells or culture of pluripotent stem cells include cells in which less than about 25%, 20%, 15%, 10%, 5% or less (e.g., 4%, 4%, 2%, 1%) of the cells express a particular phenotypic marker. In various aspects, in a population, plurality of pluripotent stem cells or culture of pluripotent stem cells, 25%, 20%, 15%, 10%, 5% or less (e.g., 4%, 4%, 2%, 1%) of the cells express a marker selected from CD34, alpha myosin, insulin, CD45 and CD133.

Pluripotent stem cells of the invention (or progeny) include co-cultures and mixed populations. Such co-cultures and mixed cell populations cells include a first mammalian (e.g., a human pluripotent stem) cell, and a second cell distinct from the first cell. A second cell can comprise a population of cells. Non-limiting examples of exemplary cells distinct from mammalian (e.g., a human pluripotent stem) cell include a T cell, dendritic cell, NK cell, monocyte, macrophage or PBMCs. Additional non-limiting examples of exemplary cells distinct from mammalian (e.g., a human pluripotent stem) cell include different adult or embryonic stem cells; totipotent, pluripotent or multipotent stem cell or progenitor or predcursor cells; cord blood stem cells; placental stem cells; bone marrow stem cells; amniotic fluid stem cells; neuronal stem cells; circulating peripheral blood stem cells; mesenchymal stem cells; germinal stem cells; adipose tissue derived stem cells; exfoliated teeth derived stem cells; hair follicle stem cells; dermal stem cells; parthenogenically derived stem cells; reprogrammed stem cells; side population stem cells; and differentiated cells.

Exemplary embryonic stem cells may express one or more antigens selected from stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT). Non-embryonic stem cells, which may be derived from cord blood stem cells, possess multipotent properties and are capable of differentiating into endothelial, smooth muscle, and neuronal cells. Cord blood stem cells may be identified based on expression of one or more antigens selected from a group comprising: SSEA-3, SSEA-4, CD9, CD34, c-kit, OCT-4, Nanog, and CXCR-4, or absence of expression of one or more markers selected from CD3, CD34, CD45, and CD11b. Such co-cultures may provide synergy between stem cell or progenitor cell populations, and may be used in the methods of the invention set forth herein.

Presence or absence of a given phenotypic marker can be determined using the methods disclosed herein (see, for example, Example 6). Thus, presence or absence of a given phenotypic marker can be determined by an antibody that binds to the marker. Accordingly, marker expression can be determined by an antibody that binds to each of the respective markers, such as CD29, CD41a, CD44, CD90, CD 105, CD34, alpha myosin, insulin or albumin, etc., in order to indicate which or how many how stem cells in a given population, plurality or culture of cells express the marker. Additional methods of detecting these and other phenotypic markers are known to one of skill in the art.

As used herein, a “cell culture” refers to the maintenance or growth of one or more cells in vitro or ex vivo. Thus, a pluripotent stem cell culture is one or mor cells in a growth medium of some kind. A “culture medium” or “growth medium” are used interchangeably herein to mean any substance or preparation used for sustaining or maintaining cells.

Cell cultures of pluripotent stem cells can take on a variety of formats. For instance, an “adherent culture” refers to a culture in which cells in contact with a suitable growth medium are present, and can be viable or proliferate while adhered to a substrate. Likewise, a “continuous flow culture” refers to the cultivation of cells in a continuous flow of fresh medium to maintain cell viability, e.g. growth.

Mammalian (e.g. human) pluripotent stem cells include individual cells, and populations and pluralities of cells (or progeny), that are isolated or purified. As used herein, the terms “isolated” or “purified” refers to made or altered “by the hand of man” from the natural state i.e. when it has been removed or separated from one or more components of the original natural in vivo environment. An isolated composition can but need not be substantially separated from other biological components of the organism in which the composition naturally occurs. An example of an isolated cell would be a pluripotent stem cell obtained from a subject such as a human. “Isolated” also refers to a composition, for example, a pluripotent stem cell separated from one or more contaminants (i.e. materials an substances that differ from the cell). A population, plurality or culture of pluripotent stem cells (or progeny) is typically substantially free of cells and materials with which it is be associated in nature.

The term “purified” refers to a composition free of many, most or all of the materials with which it typically associates with in nature. Thus, a pluripotent stem cell is considered to be substantially purified when separated from other menstrual components. Purified therefore does not require absolute purity. Furthermore, a “purified” composition can be combined with one or more other molecules. Thus, the term “purified” does not exclude combinations of compositions. Purified can be at least about 50%, 60% or more by numbers or by mass. Purity can also be about 70% or 80% or more, and can be greater, for example, 90% or more. Purity can be less, for example, in a pharmaceutical carrier the amount of a cells or molecule by weight % can be less than 50% or 60% of the mass by weight, but the relative proportion of the cells or molecule compared to other components with which it is normally associated with in nature will be greater. Purity of a population or composition of cells can be assessed by appropriate methods that would be known to the skilled artisan.

A primary isolate of a pluripotent stem cell of the invention can originate from or be derived from endometrium, endometrial stroma, endometrial membrane, or menstrual blood. Progeny of primary isolate pluripotent stem cells, which include all descendents of the first, second, third and any and all subsequent generations and cells taken or obtained from a primary isolate, that maintain stemness (e.g., phenotypic marker expression profile, doubling time, morphology, secretion of proteins, etc.) can be obtained from a primary isolate or subsequent expansion of a primary isolate. Subsequent expansion results in progeny pluripotent stem cells that can in turn comprise the populations or pluralities of stem cells, the cultures of stem cells, co-cultures, etc. Thus, a pluripotent stem cell of the invention refers to a cell from a primary isolate from endometrium, endometrial stroma, endometrial membrane, or menstrual blood, and any progeny cell therefrom. The term “derived” or “originates,” when used in reference to a pluripotent stem cell therefore means that the cells or parental cells of any previous generation at one point in time originated from endometrium, endometrial stroma, endometrial membrane, or menstrual blood. Accordingly, pluripotent stem cells are not limited to those from a primary isolate, but can be any subsequent progeny thereof or any subsequent doubling of the progeny thereof provided that the cell has the desired phenotypic markers, doubling time, or any other characteristic feature set forth herein.

Mammalian (e.g. human) pluripotent stem cells (and progeny) include those transfected with a nucleic acid. Such nucleic acids can encode proteins, polypeptides and peptides, for example, proteins, polypeptides and peptides to substitute for defectiveness, absence or deficiency of endogenous protein, polypeptide or peptide in a subject.

The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleotides. Nucleic acids include single, double or triplex, circular or linear, molecules. Exemplary nucleic acids include RNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 20 nucleotides to 20 Kb, or any numerical value or range within or encompassing such lengths, 10 nucleotides to 10Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. Shorter polynucleotides are commonly referred to as “oligonucleotides” or “probes” of single- or double-stranded DNA.

Exemplary nucleic acids encode hemoglobin, and pluripotent stem cells transfected with such a nucleic acid (or progeny) can be used to treat sickle cell anemia or alpha or beta thalassemia (hemoglobin alpha or beta chains). Another exemplary nucleic acid encodes cystic fibrosis transmembrane conductance regulator (CFTCR) protein, and pluripotent stem cells transfected with such a nucleic acid (or progeny) can be used to treat cystic fibrosis. An additional exemplary nucleic acid encodes hexosaminidase A, and pluripotent stem cells transfected with such a nucleic acid (or progeny) can be used to treat Tay Sachs disease.

A further exemplary nucleic acid encodes one or more of five gene products have been reported to form a nuclear complex, leading to the ubiquitination of a FA protein (D2), and pluripotent stem cells transfected with such a nucleic acid (or progeny) can be used to treat Fanconi anemia (FA). Another exemplary nucleic acid encodes X-linked E1 alpha gene, and pluripotent stem cells transfected with such a nucleic acid (or progeny) can be used to treat Pyruvate dehydrogenase complex deficiency (PDCD). Yet another exemplary nucleic acid encodes aldolase B, and pluripotent stem cells transfected with such a nucleic acid (or progeny) can be used to treat Congenital fructose intolerance. Still another exemplary nucleic acid encodes galactose-1 phosphate uridyl transferase, galactose kinase, or galactose-6-phosphate epimerase, and pluripotent stem cells transfected with such a nucleic acid (or progeny) can be used to treat Galactosemia.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

Nucleic acids can be included within vectors as cell transfection typically employs a vector. The term “vector,” refers to, e.g., a plasmid, virus, such as a viral vector, or other vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides in operable linkage with a nucleic acid, and expressing the transcribed encoded protein in cells in vitro, ex vivo or in vivo.

A vector generally contains at least an origin of replication for propagation in a cell. Control elements, including expression control elements, present within a vector, are included to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

Vectors can include a selection marker. A “selection marker” or equivalent means a gene that allows the selection of cells containing the gene.

“Positive selection” refers to a process whereby only cells that contain the positive selection marker will survive upon exposure to the positive selection agent or be marked. For example, drug resistance is a common positive selection marker; cells containing the positive selection marker will survive in culture medium containing the selection drug, and those which do not contain the resistance gene will die. Suitable drug resistance genes are neo, which confers resistance to G418, or hygr, which confers resistance to hygromycin, and puro which confers resistance to puromycin, among others. Other positive selection marker genes include genes that allow the identification or screening of cells. These genes include genes for fluorescent proteins, the lacZ gene, the alkaline phosphatase gene, and surface markers such CD8, among others.

“Negative selection” refers to a process whereby cells containing a negative selection marker are killed upon exposure to an appropriate negative selection agent which kills cells containing the negative selection marker. For example, cells which contain the herpes simplex virus-thymidine kinase (HSV-tk) gene are sensitive to the drug gancyclovir (GANC). Similarly, the gpt gene renders cells sensitive to 6-thioxanthine.

Vectors included are those based on viral vectors, such as retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Simian virus 40 (SV40) and bovine papilloma virus (BPV) have the ability to replicate as extra-chromosomal elements (Eukaryotic Viral Vectors, Cold Spring Harbor Laboratory, Gluzman ed., 1982; Sarver et al., Mol. Cell. Biol. 1:486 (1981)). Additional viral vectors useful for expression include reovirus, parvovirus, Norwalk virus, coronaviruses, paramyxo- and rhabdoviruses, togavirus (e.g., sindbis virus and semliki forest virus) and vesicular stomatitis virus (VSV) for introducing and directing expression of a polynucleotide or transgene in pluripotent stem cells or progeny thereof (e.g., differentiated cells).

Vectors including a nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. For expression in mammalian cells, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

In accordance with the invention, there are provided pluripotent stem cells transfected with a nucleic acid or vector. Such transfected cells include but are not limited to a primary cell isolate, populations or pluralityes of pluripotent stem cells, cell cultures (e.g., passaged, established or immortalized cell line), as well as progeny cells thereof (e.g., a progeny of a transfected cell that is clonal with respect to the parent cell, or has acquired a marker or other chartacteristic of differentiation).

The term “transfected” when use in reference to a cell (e.g., a host pluripotent stem cell), means a genetic change in a cell following incorporation of an exogenous molecule, for example, a nucleic acid (e.g., a transgene) or protein into the cell. Thus, a “transfected” cell is a cell into which, or a progeny thereof in which an exogenous molecule has been introduced by the hand of man, for example, by recombinant DNA techniques.

The nucleic acid or protein can be stably or transiently transfected (expressed) in the cell and progeny thereof. The cell(s) can be propagated and the introduced nucleic acid transcribed and protein expressed. A progeny of a transfected cell may not be identical to the parent cell, since there may be mutations that occur during replication.

Viral and non-viral vector means of delivery into pluripotent stem cells, in vitro, in vivo and ex vivo are included. Introduction of compositions (e.g., nucleic acid and protein) into target cells (e.g., host pluripotent stem cells) can be carried out by methods known in the art, such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.”

Pluripotent stem cells of the invention (or progeny) including pluripotent stem cell populations, pluralities of pluripotent stem cells, cultures of pluripotent stem cells (cell cultures) and co-cultures and mixed populations can be sterile, and maintained in a sterile environment. Such cells, pluralities, populations, and cultures thereof can also be included in a medium, such as a liquid medium suitable for administration to a subject (e.g., a mammal such as a human).

Methods for producing a pluripotent stem cell are provided. In one embodiment, a method includes obtaining a menstrual blood sample, cloning one or more cells from the sample, selecting one or more cells based upon morphology or growth rate or phenotypic marker expression profile, thereby isolating a pluripotent stem cell.

Methods for producing populations and pluralities of pluripotent stem cells are also provided. In such methods, expanding pluripotent stem cells for a desired number of cell divisions (doublings) thereby produces increased numbers or a population or plurality of pluripotent stem cells. Relative proportions or amounts of pluripotent stem cells within cell cultures include 50%, 60%, 70%, 80%, 90% or more pluripotent stem cells in a population or plurality of cells.

Methods for producing a culture of pluripotent stem cells are further provided. In one embodiment, a method includes providing one or more pluripotent stem cells based upon morphology or growth rate or phenotypic marker expression, and placing said cells in contact with a culture medium, thereby producing a culture of pluripotent stem cells. Cells of such cell cultures can optionally be expanded.

The term “contact,” when used in reference to cells, a population of cells or a cell culture or a method step or treatment, means a direct or indirect interaction between the composition (e.g., cell or cell culture) and the other referenced entity. A particular example of a direct interaction is physical interaction. A particular example of an indirect interaction is where a composition acts upon an intermediary molecule which in turn acts upon the referenced entity (e.g., cell or cell culture).

Methods for producing a differentiated progeny cell (e.g., a progenitor cell, a precursor cell, a developmental intermediate or ultimately differentiated cell) from a pluripotent stem cell are further provided. In one embodiment, a method includes culturing one or more pluripotent stem cells under conditions that facilitate differentiation of the cell or cells to a progenitor cell, a precursor cell, or a developmental intermediate of an adipogenic, endothelial, hepatic, osteogenic, pancreatic, neural or myocytic cell, or an ultimately differentiated adipogenic, endothelial, hepatic, osteogenic, pancreatic, neural or myocytic cell.

In a specific embodiment, methods of producing pancreatic islets, or insulin-producing cells from pluripotent stem cells are provided. To illustrate, a culture of pluripotent stem cells is treated with a serum-free, low-glucose medium containing dimethyl sulfoxide (e.g., 5.5 mM glucose and 1% DMSO). This culture step can prime the cells for further differentiation into endocrine hormone-producing (e.g., insulin-secreting) cells (see, e.g., U.S. Pat. No. 7,169,608). Pluripotent stem cells may be cultured in this low-glucose medium for approximately 3 days (e.g., 1 to 5 days) in a media such as DMEM. Following this initial culture step, pluripotent stem cells are subsequently exposed to a high-glucose medium containing serum. This second culture is differentiates the pluripotent stem cells into endocrine hormone-producing cells. The second culture is approximately 7 days. The high concentration of glucose is approximately 25 mM. The concentration of serum is approximately 10%. Numerous types of serum may be used including human, fetal calf serum, or cord blood serum. Quality of insulin producing cells may be detected morphologically, by ability of differentiated cells to self-assemble to form three-dimensional islet cell-like clusters, as well as expression of pancreatic islet cell differentiation-related transcripts detectable by reverse transcription-PCR/nested PCR such as PDX-1, PAX-4, PAX-6, Nkx2.2 and Nkx6.1, insulin I, insulin II, glucose transporter 2, and glucagons. Hormones produced that indicate that the cells are truly similar to islets or only produce insulin include glucagon, and pancreatic polypeptide, which may be detected by immunohistochemistry, Yang, e.t al., Proc. Nat.l Acad. Sci. U.S.A. 99:8078 (2002).Other agents may be added to this culture system for increasing the concentration of insulin producing cells, such as nicotinamide, Otonkoski, et al., J. Clin. Invest. 92:1459 (1993); polyamines, Sjoholm, et. al., Endocrinology 135:1559 (1994); hepatocyte growth factor Beattie, et al. Diabetes 45:1223 (1996); and, betacellulin, Cho, et. al., Biochem. Biophys. Res. Commun. 366:129 (2008). Various extracellular matrix components such as fibronectin and laminin may also be added to increase yield or concentration of islets/insulin-producing cells, Leite, et al., Cell Tissue Res. 327:529 (2008).

Ability of the cells to function in vivo may be studied using animal models or in clinical trials. A commonly used model involves administration of putative insulin producing cells into mice that have been treated with streptozoticin, which destroys insulin producing beta-cells. Recipient mice may be immune suppressed or immune deficient, such as nude mice, RAG knockout, or SCID mice. Production of human C-peptide may be used as a proxy of insulin production, alternatively glucose responsiveness may be studied. An example of in vivo assessment of stem cell derived insulin producing cells is provided in Davani, et al., Stem Cells 25:3215 (2007).

Methods for increasing, stimulating, inducing, promoting, augmenting or enhancing proliferation or differentiation of a totipotent, pluripotent or multipotent stem cell, or a progenitor or precursor cell, or a differentiated cell, in vitro, ex vivo and in vivo cell are provided. In various embodiments, methods include co-culturing (contacting) a pluripotent stem cell, or a population or plurality of pluripotent stem cells (or progeny), and a totipotent, pluripotent or multipotent stem cell, or a progenitor or precursor cell, or a differentiated cell, thereby stimulating, inducing, promoting, augmenting or enhancing proliferation or differentiation of the totipotent, pluripotent or multipotent stem cell, or a progenitor or precursor cell, or a differentiated cell. In a particular embodiment, a method includes co-culturing (contacting) a human pluripotent stem cell, or a population or plurality of cells, and PBMCs, or a cell present in or derived from PBMCs (e.g., a CD4+ T cell or an NK T cell), under conditions facilitating increased numbers of T regulatory cells, thereby increasing numbers of T regulatory cells. In another particular embodiment, a method of increasing numbers of T regulatory cells in a subject includes administering a human pluripotent stem cell, or or a population or plurality of cells, to a subject under conditions facilitating increased numbers of T regulatory cells.

Any of the foregoing method steps can optionally include isolating the one or more pluripotent stem cells (or progeny), and optionally include purifying the one or more pluripotent stem cells (or progeny). Thus, in accordance with the invention, methods of isolating the one or more pluripotent stem cells (or progeny), and purifying the one or more pluripotent stem cells (or progeny) are provided.

Any of the foregoing method steps can optionally include expanding the one or more pluripotent stem cells (or progeny) for one or more cell divisions (doublings). Thus, in accordance with the invention, methods of increasing numbers of the mammalian (e.g., human) pluripotent stem cell (or progeny) are provided. In one embodiment, a method includes culturing a mammalian (e.g., human) pluripotent stem cell (or differentiated progeny) in a growth medium under conditions allowing the cells to proliferate. In particular aspects, the cells proliferate or increase in numbers with less than 25%, 20%, 15%, 10%, 5% or less of the cells undergoing transformation, exhibiting karyotype variations, or differentiating. In additional aspects, the cells are cultured in a serum-free medium capable of maintaining cellular viability, the cells are cultured under anaerobic conditions or conditions of hypoxia, and the cells are cultured in the presence of a compound capable of upregulating a cell regenerative activity.

Pluripotent stem cells, populations and pluralities of pluripotent stem cells, pluripotent stem cell cultures and differentiated progeny can be kept or maintained for a period of time (e.g., 1-24 minutes, hours, days, weeks, etc.), can be expanded, or can be allowed to progress to a subsequent developmental, maturation or differentiation stage. Any of the foregoing method steps can optionally include clonal expansion or maturation or differentiation pluripotent stem cells.

Pluripotent stem cells, populations and pluralities of pluripotent stem cells, differentiated progeny and methods for expanding, isolating or producing, can include growth medium, which can be added or changed at any time, for a period of 1-60 minutes, 1-60 hours or 1-60 days. In exemplary embodiments, fresh growth media is added every 24-48 hours, or during passaging or expanding the cells or following a step of a method of the invention. In additional exemplary embodiments, fresh growth media is added to a pluripotent stem cells (or differentiated progeny) at a given developmental, maturation or differentiation stage, or during cell expansion (proliferation).

During growth, culture or expansion of pluripotent stem cells, populations or a plurality of pluripotent stem cells, co-cultures or a mixed population of pluripotent stem cells, or progeny differentiated cells of any developmental, maturation or differentiation stage, other factors which stimulate cellular metabolism, division, growth (proliferation) and optionally differentiation can be added to enrich (increase numbers) of pluripotent stem cells or facilitate differentiation of pluripotent stem cells in vitro or ex vivo or in vivo. Non limiting examples of factors include EPO, TPO, flt-3 ligand, stem cell factor, M-CSF, G-CSF, GM-CSF, IL-3, IL-6, IL-7, TGF-b, PDGF, FGF, VEGF, and PIGF. Angiogenic agents include, for example, cytokines such as EGF, VEGF, FGF, EGF, and angiopoietin.

Pluripotent stem cells, including individual clones, populations, pluralities and cultures of pluripotent stem cells, differentiated progeny and methods for producing pluripotent stem cells, including individual clones, populations, pluralities and cultures of pluripotent stem cells include cells produced by a treatment that includes hypoxia or anaerobic conditions so that cells unable to survive by anaerobic metabolism senesce or die are provided, thereby enriching for cells that survive via anaerobic metabolism. Pluripotent stem cells, including individual clones, populations, pluralities and cultures of pluripotent stem cells and methods for producing pluripotent stem cells, including individual clones, populations, pluralities and cultures of pluripotent stem cells include conditions of reduced oxygen (e.g., less than 2%), such as hypoxia, or contact with lactic acid.

Pluripotent stem cells, including individual clones, populations, pluralities and cultures of pluripotent stem cells, and differentiated progeny can be distributed in a vessel or container such as a dish (single or multiwell), plate (single or multiwell), vial, tube, bottle (e.g., roller bottle), flask, bag, syringe or jar. Multi-well dishes and plates include an 8, 16, 32, 64, 96, 384 and 1536 multi-well dish or plate. Pluripotent stem cells, including individual clones, populations, pluralities and cultures of pluripotent stem cells, and differentiated progeny can be attached to a substrate, such as a slide, a dish (single or multiwell), plate (single or multiwell), vial, tube, bottle, or flask.

The invention further provides conditioned medium and methods of producing conditioned medium. A conditioned medium is or has been in contact with (e.g., incubated) which a particular cell or population of cells for a period of time, and then removed, and thus can be produced accordingly. While the cells are cultured in the medium, they secrete cellular factors into the medium, such as matrix metalloprotease 3 (MMP3), matrix metalloprotease 10 (MMP10), GM-CSF, PDGF-BB or angiogenic factor ANG-2, but are not limited to these particular factors and may secrete additional factors. The medium containing these alone or in combination with other factors is the conditioned medium. In various embodiments, a medium has been incubated with a pluripotent stem cell or population, plurality or culture, or co-culture, for a period of about 1-72 hours, 3-7 days, or more. In particular aspects, the medium includes one or more of matrix metalloprotease 3 (MMP3), matrix metalloprotease 10 (MMP10), GM-CSF, PDGF-BB or angiogenic factor ANG-2. In various aspects, the medium stimulates, increases, induces, promotes, enhances or augments cell survival, viability, growth, proliferation or differentiation of a totipotent stem cell, a pluripotent stem cell, a multipotent stem cell or a differentiated cell. In additional various aspects, the medium stimulates, increases, induces, promotes, enhances or augments cell survival, viability, growth, proliferation or differentiation of a human umbilical vein endothelial cell.

Conditioned medium and methods of producing conditioned medium additionally include concentrated (concentrating), lyophilized (lyophilizing) and freeze-dried forms (freeze drying). Such medium can be separated from cells by withdrawal from a cell culture, such as by aspiration or dispensing the medium, in a container or vessel.

Pluripotent stem cells, populations and pluralities of pluripotent stem cells, cell cultures of pluripotent stem cells, and conditioned medium include storing, stored, preserving and preserved pluripotent stem cells and conditioned medium. In various embodiments, storing, stored, preserving and preserved pluripotent stem cells and conditioned medium include freezing (frozen) or storing (stored) pluripotent stem cells and conditioned medium, such as, for example, individual pluripotent stem cell clones, a population or plurality of pluripotent stem cells, a culture of pluripotent stem cells, co-cultures and mixed populations of pluripotent stem cells and other cell types and conditioned medium. Pluripotent stem cells and conditioned medium can be preserved or frozen, for example, under a cryogenic condition, such as at −20 degrees C. or less, e.g., −70 degrees C. Preservation or storage under such conditions can include a membrane or cellular protectant, such as dimethylsulfoxide (DMSO).

Mammalian (e.g. human) pluripotent stem cells, a population or plurality or culture of pluripotent stem cells, progeny of pluripotent stem cells (e.g., any clonal progeny or any or all various developmental, maturation and differentiation stages) and conditioned medium of pluripotent stem cells can be used for various applications, can be used in accordance with the methods of the invention including treatment and therapeutic methods. The invention therefore provides in vivo and ex vivo treatment and therapeutic methods that employ mammalian (e.g. human) pluripotent stem cells, populations and pluralities and cultures of pluripotent stem cells, progeny of pluripotent stem cells and conditioned medium of pluripotent stem cells.

Pluripotent stem cells, a population or plurality or culture of pluripotent stem cells, progeny of pluripotent stem cells and conditioned medium of pluripotent stem cells can be administered to a subject, or used to implant or transplant as a cell-based or medium based therapy, or to provide factors, such as secreted MMPs or other cytokines (e.g., GMCSF, PDGF-BB, or angiogenic factor ANG-2) to provide a benefit to a subject (e.g., by differentiating into cells in the subject, or stimulate, increase, induce, promote enhance or augment activity or function of endogenous stem cells or endogenous differentiated cells). Cells and conditioned medium can be collected from a population or plurality or culture of pluripotent stem cells, e.g., after the initial cloning and during optional expansion phase of pluripotent stem cells.

In accordance with the invention, methods of providing a stem cell therapy and methods of treating a subject that would benefit from a stem cell therapy are provided. In one embodiment, a method includes administering pluripotent stem cells, a population or plurality or culture of pluripotent stem cells, progeny of pluripotent stem cells or conditioned medium of pluripotent stem cells to the subject in an amount sufficient to provide a benefit to the subject. In particular non-limiting aspects, a subject is in need of increased, stimulated, induced, promoted, augmented or enhanced hematopoiesis. In additional non-limiting aspects, a subject is in need of increased, stimulated, induced, promoted, augmented or enhanced liver function or activity; in need of reduced, decreased, inhibited, blocked, prevented, controlled or limited inflammation or autoimmunity; or in need of increased, stimulated, induced, promoted, augmented or enhanced angiogenesis.

Thus, methods of the invention include administering pluripotent stem cells, a population or plurality or culture of pluripotent stem cells, progeny of pluripotent stem cells or conditioned medium of pluripotent stem cells to increase, stimulate, induce, promote, augment or enhance hematopoiesis (in a deficient subject); to increase, stimulate, induce, promote, augment or enhance liver function or activity; to reduce, decrease, inhibit, block, prevent, control or limit inflammation (e.g., to a subject in need of inhibition of inflammation); and to increase, stimulate, induce, promote, augment or enhance angiogenesis. For example, pluripotent stem cells can be administered (e.g., intravenously) to a subject with ischemia, so as to induce angiogenesis (e.g., by homing to ischemic tissuein the subject). Numerous diseases have been associated with ischemia, including stroke, ischemic heart disease, liver failure, kidney failure, and peripheral artery disease.

Further, methods of the invention include administering pluripotent stem cells, a population or plurality or culture of pluripotent stem cells, progeny of pluripotent stem cells or conditioned medium of pluripotent stem cells to treat a subject having or at risk of having ischemia in a tissue or organ (e.g., cardiac or pulmonary tissue, limb, or kidney); to treat a subject having or at risk of having a stroke, pulmonary fibrosis, or diabetic limb; to treat a subject in need of inhibition of fibrosis or scar tissue formation; to treat a subject having or at risk of having fibrosis or scar tissue formation in a tissue or organ (e.g., cardiac or pulmonary, limb, liver, pancreas, or kidney); to treat a subject in need of inhibition, reduction, decreased, controlled or reversal of pathological apoptosis; to treat a subject in need of increasing or improving a pancreas or liver function; to increase numbers or proliferation of islet cells, increase numbers or proliferation of hepatocytes, or increase insulin production; to treat a subject having or at risk of having diabetes, liver failure, cirhossis, liver or pancreas fibrosis, or hepatitis; to treat a subject in need of osteocytes or an osteocyte function (e.g., to increase, stimulate, induce, promote, augment or enhance osteocyte numbers, osteocyte formation or osteocyte function); to treat a subject having or at risk of having osteoporosis, a bone fracture or break, or is in need of a prosthesis in a joint; and to treat a subject in need of dermal stem cells, or activation or stimulation of endogenous dermal stem cells.

Moreover, methods of the invention include administering pluripotent stem cells, a population or plurality or culture of pluripotent stem cells, progeny of pluripotent stem cells or conditioned medium of pluripotent stem cells to treat a subject in need of increased or improved pulmonary or cardiac function, for example, a subject that has or is at risk of having a cardiac or pulmonary disease. Non-limiting examples of cardiac and pulmonary diseases include artherosclerosis, myocardial infarction (Heart Attack), cardiac infection, heart failure, ischemic heart failure, high blood pressure (Hypertension), or pulmonary hypertension, idiopathic pulmonary fibrosis, stroke, congenital heart disease (CHD), congestive heart failure, angina, myocarditis, coronary artery disease, cardiomyopathy, dilated cardiomyopathy, hypertrophic cardiomyopathy, endocarditis, diastolic dysfunction, cerebrovascular disease, valve disease, mitral valve prolapse, venous thromboembolism or arrhythmia.

Additionally, methods of the invention include administering pluripotent stem cells, a population or plurality or culture of pluripotent stem cells, progeny of pluripotent stem cells or conditioned medium of pluripotent stem cells to treat a subject having or at risk of having a neurological or muscular disease or disorder. Non-limiting examples of neurological and muscular diseases and disorders include multiple sclerosis (MS), spinal cord injury, muscular dystrophy (Becker's or Duchenne's), amyotrophic lateral sclerosis (ALS; Lou Gehrig's disease or classical motor neuron disease), autism, progressive bulbar palsy (progressive bulbar atrophy), pseudobulbar palsy, primary lateral sclerosis (PLS), progressive muscular atrophy, spinal muscular atrophy (SMA, including SMA type I—Werdnig-Hoffmann disease, SMA type II, or SMA type III—Kugelberg-Welander disease), Fazio-Londe disease, Kennedy disease (progressive spinobulbar muscular atrophy), congenital SMA with arthrogryposis, and post-polio syndrome (PPS).

Methods of the invention also include administering pluripotent stem cells, a population or plurality or culture of pluripotent stem cells, progeny of pluripotent stem cells or conditioned medium of pluripotent stem cells to treat a subject having or at risk of having an immune or inflammatory mediated disorder or disease, such as an autoimmune disease or disorder Non-limiting examples include: Thyroiditis, insulitis, multiple sclerosis, iridocyclitis, uveitis, orchitis, Addison's disease, myasthenia gravis, rheumatoid arthritis, lupus erythematosus, immune hyperreactivity, insulin dependent diabetes mellitus, anemia (aplastic, hemolytic), hepatitis, autoimmune hepatitis, skleritis, idiopathic thrombocytopenic purpura, diseases of the gastrointestinal tract (e.g., Crohn's disease, ulcerative colitis and other inflammatory bowel diseases), juvenile arthritis, scleroderma and systemic sclerosis, sjogren's syndrom, undifferentiated connective tissue syndrome, antiphospholipid syndrome, vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, including rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, vascular inflammatory disorders, respiratory distress syndrome or other cardiopulmonary diseases, inflammation associated with peptic ulcer, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the preventing or treating graft rejection in cases of transplantation of natural or artificial cells, tissue and organs such as liver, kidney, heart, lung, cornea, bone marrow, organs, lenses, pacemakers, natural or artificial skin tissue.

Still further, methods of the invention include administering pluripotent stem cells, a population or plurality or culture of pluripotent stem cells, progeny of pluripotent stem cells or conditioned medium of pluripotent stem cells to a subject in need of stimulating, increased, inducing, augmenting, or enhanced immunological tolerance. Such methods can stimulate, increase, induce, augment, or enhance immunological tolerance thereby treating an autoimmune disorder.

Still moreover, methods of the invention include administering pluripotent stem cells, a population or plurality or culture of pluripotent stem cells, progeny of pluripotent stem cells or conditioned medium of pluripotent stem cells to a subject in need of inhibiting, reducing, decreasing, blocking, preventing, controlling or limiting immunological rejection of a transplant, transplant fibrosis or graft failure. Such methods can inhibit, reduce, decrease, block, prevent, control or limit immunological rejection of the transplant, transplant fibrosis or graft failure thereby enhancing acceptance of the transplant or graft by the subject.

Still additionally, methods of the invention include administering pluripotent stem cells, a population or plurality or culture of pluripotent stem cells, progeny of pluripotent stem cells or conditioned medium of pluripotent stem cells to treat a subject in need of treatment for a melanoma. As disclosed in Example 14, 2 of 3 mice afflicted with a melanoma responded to treatment with pluripotent stem cells, whereas all control mice were afflicted with melanoma

Pluripotent stem cells, a population or plurality or culture of pluripotent stem cells, progeny of pluripotent stem cells or conditioned medium of pluripotent stem cells can be administered or delivered to a subject by any route suitable for the treatment method or protocol. Specific non-limiting examples of administration and delivery routes include parenteral, e.g., intravenous, intramuscular, intrathecal (intra-spinal), intrarterial, intradermal, subcutaneous, intra-pleural, transdermal (topical), transmucosal, intra-cranial, intra-ocular, mucosal, implantation and transplantation.

Pluripotent stem cells, a population or plurality or culture of pluripotent stem cells, progeny of pluripotent stem cells or conditioned medium of pluripotent stem cells can be autologous with respect to the subject, that is, the stem cells used in the method (or to produce the conditioned medium) were obtained or derived from a cell from the subject that is treated according to the method. Pluripotent stem cells, a population or plurality or culture of pluripotent stem cells, progeny of pluripotent stem cells or conditioned medium of pluripotent stem cells can be allogeneic with respect to the subject, that is, the stem cells used in the method (or to produce the conditioned medium) were obtained or derived from a cell from a subject that is different from the subject that is treated according to the method.

Methods of the invention include administering pluripotent stem cells, a population or plurality or culture of pluripotent stem cells, progeny of pluripotent stem cells or conditioned medium of pluripotent stem cells prior to concurrently with or following administration of additional pharmaceutical agents or biologics. Pharmaceutical agents or biologics may activate or stimulate stem cells. Non-limiting examples of such agents include, for example: erythropoietin Tsai, e. t al., J. Neurosci. 26:1269 (2006); prolactin, Ogueta, et. al. Mol. Cell Endocrinol. 190:51 (2002); human chorionic gonadotropin (U.S. Pat. No. 5,968,513); gastrin, Banerjee et. al. Rev. Diabet. Stud. 2:165 (2005); EGF, Brand, et. al., Pharmacol Toxicol 91:414 (2002); FGF, Wang, et. al., Am. J. Physiol. Heart Circ. Physiol 286:H1985 (2004); and/or, VEGF, Yildirim, et. al., Bone Marrow Transplant 36:71 (2005). Pharmaceutical agents or biologics may inhibit or reduce an activity or function of stem cells. For example, inhibitors (neutralizers) of TNF alpha may be administered prior to concurrently with or following administration of stem cells to de-repress inhibitory effects of this cytokine on circulating stem cells, Ablin, et. al., Life Sci 79:2364 (2006).

Pharmaceutical agents also include anti-inflammatory agents. Non-limiting examples of anti-inflammatory include Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium.

Methods of the invention include, among other things, methods that provide a detectable or measurable improvement in a condition of a given subject, such as alleviating or ameliorating one or more adverse (physical) symptoms or consequences associated with the presence of a disease, disorder, illness, pathology, or an adverse symptom, effect or complication caused by or associated with the disease, disorder, illness, pathology, i.e., a therapeutic benefit or a beneficial effect.

A therapeutic benefit or beneficial effect is any objective or subjective, transient, temporary, short-term or long-term improvement in the a disease, disorder, illness, or pathology, or a reduction in onset, severity, duration or frequency of an adverse symptom associated with or caused by a disease, disorder, illness, or pathology. A satisfactory clinical endpoint of a treatment method in accordance with the invention is achieved, for example, when there is an incremental or a partial reduction in severity, duration or frequency of one or more associated adverse symptoms, effects or complications of a disease, disorder, illness, or pathology, or inhibition or reversal of one or more of the physiological, biochemical or cellular manifestations or characteristics of the disease, disorder, illness, or pathology. A therapeutic benefit or improvement therefore be a cure or ablation of one or more, most or all adverse symptoms, effects or complications associated with or caused by a disease, disorder, illness, or pathology. However, a therapeutic benefit or improvement need not be a cure or complete ablation of all pathologies, adverse symptoms, effects or complications associated with or caused by the disease, disorder, illness, or pathology.

The terms “subject” and “patient” are used interchangeably herein and refer to animals, typically mammals, such as humans, non-human primates (gorilla, chimpanzee, orangutan, macaque, gibbon), domestic animals (dog and cat), farm and ranch animals (horse, cow, goat, sheep, pig), laboratory and experimental animals (mouse, rat, rabbit, guinea pig). Human subjects include children, for example, newborns, infants, toddlers and teens, between the ages of 1 and 5, 5 and 10 and 10 and 18 years, adults between the ages of 18 and 60 years, and the elderly, for example, between the ages of 60 and 65, 65 and 70 and 70 and 100 years.

Subjects include those that are likely to benefit from treatment with pluripotent stem cells, populations, pluralities or cultures of pluripotent stem cells, progeny and cells differentiated therefrom. Subjects include those that are likely to benefit from culture medium conditioned or factors produced therefrom, or new cells or new tissue, stimulation of endogenous progenitor cell proliferation, stimulation of endogenous stem cell proliferation, stimulation of endogenous progenitor cell differentiation, or stimulation of endogenous stem cell differentiation. Accordingly, subjects include mammals (e.g., humans) in need of treatment or that would benefit from a stem cell treatment, or treatment with progeny or cells differentiated from pluripotent stem cells, or culture medium conditioned or factors produced by pluripotent stem cells, or progeny cells such as cells differentiated therefrom.

Non-limiting exemplary subjects for treatment include those that would benefit from of increased, stimulated, induced, promoted, augmented or enhanced angiogenesis, hemtaopoiesis or liver function or activity. Additional non-limiting exemplary subjects for treatment include those that would benefit from endogenous progenitor cell proliferation, endogenous stem cell proliferation, endogenous progenitor cell differentiation endogenous stem cell differentiation, exogenous progenitor cell proliferation, exogenous stem cell proliferation exogenous progenitor cell differentiation or exogenous stem cell differentiation

Further non-limiting exemplary subjects for treatment include those that would benefit from reducing, decreasing, inhibiting, controlling, limiting, blocking or preventing fibrosis or scar tissue formation; reducing, decreasing, inhibiting, controlling, limiting, blocking or preventing inflammation or an autoimmune disorder; or reducing, decreasing, inhibiting, controlling, limiting, blocking or preventing undesired or pathological apoptosis.

Still additional non-limiting exemplary subjects for treatment include those that would benefit from increased numbers or improved function, healing or repair of adipogenic, endothelial, hepatic, osteogenic, pancreatic, neural or myocytic cells, comprising administering adipogenic, endothelial, hepatic, osteogenic, pancreatic, neural or myocytic cells, whether it be the subjects own (endogenous) adipogenic, endothelial, hepatic, osteogenic, pancreatic, neural or myocytic organ or tissue, or an exogenously provided cells (e.g., pluripotent stem cells, or progeny thereof).

Subjects yet additionally include include those having or at risk of having diabetes, liver failure, a neurological dirorder or disease, or lung fibrosis. Subjects also include those at risk of having a cardiac disease or disorder. Target subjects for treatment therefore include those having or at risk of having a cardiac disease or disorder. Exemplary cardiac diseases and disorders included, but are not limited to, atherosclerosis, stroke, congenital heart disease, congestive heart failure, angina, myocarditis, coronary artery disease, cardiomyopathy, dilated cardiomyopathy, hypertrophic cardiomyopathy, endocarditis, myocardial infarction (Heart Attack), diastolic dysfunction, cerebrovascular disease, valve disease, high blood pressure (Hypertension), mitral valve prolapse and venous thromboembolism.

At risk subjects include those with a family history (high blood pressure, heart disease), genetic predisposition (hypercholesterolemia), or who have suffered a previous affliction with a cardiac disease or disorder. At risk subjects further include those with or at risk of high blood pressure or high cholesterol due to a genetic predisposition or a diet, such as high fat, or environmental exposure, such as smokers.

A “donor” is a subject used as a source of a biological material, such as endometrium, endometrial stroma, endometrial membrane, or menstrual blood. A “recipient” is a subject which accepts a biological material. In autologous transfers, the donor and recipient are one and the same, i.e., syngeneic.

The doses or “amount effective” or “amount sufficient” in a method of treatment in which it is desired to achieve a therapeutic benefit or improvement includes, for example, any objective or subjective alleviation or amelioration of one, several or all adverse symptoms, effects or complications associated with or caused by the disease, disorder, illness, or pathology to a measurable or detectable extent. Thus, in the case of a particular a disease, disorder, illness, or pathology, the amount will be sufficient to provide a therapeutic benefit to a given subject or to alleviate or ameliorate an adverse symptom, effect or complication of the a disease, disorder, illness, or pathology in a given subject. The dose may be proportionally increased or reduced as indicated by the status of treatment or any side effect(s). Exemplary doses can be an amount of cells ranging from 500,000-500 million, typically between 1-100 million cells.

In methods of treatment, a method may be practiced one or more times (e.g., 1-10, 1-5 or 1-3 times) per day, week, month, or year. The skilled artisan will know when it is appropriate to delay or discontinue administration. Frequency of administration is guided by clinical need or surrogate markers. An exemplary non-limiting dosage schedule is every second day for a total of 4 injections, 1-7 times per week, for 1, 2, 3, 4, 5, 6, 7, 8, 9, 10, 15, 20 or more weeks, and any numerical value or range or value within such ranges.

Of course, as is typical for any treatment or therapy, different subjects will exhibit different responses to treatment and some may not respond or respond less than desired to a particular treatment protocol, regimen or process. Amounts effective or sufficient will therefore depend at least in part upon the disorder treated (e.g., the type or severity of the disease, disorder, illness, or pathology), the therapeutic effect desired, as well as the individual subject (e.g., the bioavailability within the subject, gender, age, etc.) and the subject's response to the treatment based upon genetic and epigenetic variability (e.g., pharmacogenomics).

The invention further provides kits, including pluripotent stem cells, populations or a plurality of pluripotent stem cells, cultures of pluripotent stem cells, co-cultures and mixed populations of pluripotent stem cells, progeny differentiated cells of any developmental, maturation or differentiation stage, as well as conditioned medium produced by contact with pluripotent stem cells, packaged into suitable packaging material. In various non-limiting embodiments, a kit includes a pluripotent stem cell population or culture, or a co-culture or a mixed population thereof. In various aspects, a kit includes instructions for using the kit components e.g., instructions for performing a method of the invention, such as culturing, expanding (increasing cell numbers), proliferating, differentiating, maintaining, or preserving pluripotent stem cells, or a pluripotent stem cells cell based treatment or therapy. In various aspects, a kit includes an article of manufacture, for example, an article of manufacture for culturing, expanding (increasing cell numbers), proliferating, differentiating, maintaining, or preserving pluripotent stem cells, such as a tissue culture dish or plate (e.g., a single or multi-well dish or plate such as an 8, 16, 32, 64, 96, 384 and 1536 multi-well plate or dish), tube, flask, bag, syringe, bottle or jar. In additional various aspects, a kit includes an article of manufacture, for example, an article of manufacture for administering, introducing, transplanting, or implanting pluripotent stem cells into a subject locally, regionally or systemically.

The term “packaging material” refers to a physical structure housing the components of the kit. The packaging material can be sealed or maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention. Thus, in additional embodiments, a kit includes a label or packaging insert including instructions for practicing a method of the invention in solution, in vitro, in vivo, or ex vivo. Instructions can therefore include instructions for practicing any of the methods of the invention described herein. Instructions may further include indications of a satisfactory clinical endpoint or any adverse symptoms or complications that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a tissue culture dish, tube, flask, roller bottle, plate (e.g., a single multi-well plate or dish such as an 8, 16, 32, 64, 96, 384 and 1536 multi-well plate or dish) or vial containing a component (e.g., pluripotent stem cells) of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include cell growth medium, buffering agent, a preservative, or a cell stabilizing agent. Each component of the kit can be enclosed within an individual container or in a mixture and all of the various containers can be within single or multiple packages.

Pluripotent stem cells, populations or a plurality of pluripotent stem cells, cultures of pluripotent stem cells, co-cultures or a mixed populations of pluripotent stem cells, progeny differentiated cells of any developmental, maturation or differentiation stage, as well as conditioned medium produced by contact with pluripotent stem cells can be packaged in dosage unit form for administration and uniformity of dosage. “Dosage unit form” as used herein refers to physically discrete units suited as unitary dosages; each unit contains a quantity of the composition in association with a desired effect. The unit dosage forms will depend on a variety of factors including, but not necessarily limited to, the particular composition employed, the effect to be achieved, and the pharmacodynamics and pharmacogenomics of the subject to be treated.

Pluripotent stem cells, populations or a plurality of pluripotent stem cells, cultures of pluripotent stem cells, co-cultures or a mixed populations of pluripotent stem cells, progeny differentiated cells of any developmental, maturation or differentiation stage, and conditioned medium, can be included in or employ pharmaceutical formulations. Pharmaceutical formulations include “pharmaceutically acceptable” and “physiologically acceptable” carriers, diluents or excipients. The terms “pharmaceutically acceptable” and “physiologically acceptable” mean that the formulation is compatible with pharmaceutical administration. Such pharmaceutical formulations are useful for, among other things, administration or delivery to, implantation or transplant into, a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical formulations can be made to be compatible with a particular local, regional or systemic administration or delivery route. Thus, pharmaceutical formulations include carriers, diluents, or excipients suitable for administration by particular routes. Specific non-limiting examples of routes of administration for compositions of the invention are parenteral, e.g., intravenous, intramuscular, intrathecal (intra-spinal), intrarterial, intradermal, subcutaneous, intra-pleural, transdermal (topical), transmucosal, intra-cranial, intra-ocular, mucosal administration, and any other formulation suitable for the treatment method or administration protocol.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

All patents, publications, Genbank accession numbers and other references cited herein are incorporated by reference in their entirety. In case of conflict, the present specification, including definitions, will control.

As used herein, singular forms “a”, “and,” and “the” include plural referents unless the context clearly indicates otherwise. Thus, for example, reference to a “a pluripotent stem cells or a progeny differentiated from a pluripotent stem cell,” includes a plurality of stemm cells or progeny thereof, and reference to “a cell culture” can include multiple cell types of varied developmental, maturation or differentiation stage within the culture.

As used herein, all numerical values or numerical ranges include whole integers within or encompassing such ranges and fractions of the values or the integers within or encompassing ranges unless the context clearly indicates otherwise. Thus, for example, reference to a range of 0.5-1.5, includes any numerical value or range within or encompassing such values, such as 0.6, 0.7, 0.8, 0.9, 1.0, 1.1, 1.2, 1.3 and 1.5, 0.55, 0.56, 0.57. 0.58. 0.59, etc., and any numerical range within such a range, such as 0.5-0.8, 0.8-1.0, 1.0-1.2, 1.0-1.4, 1.2-1.4, 1.3-1.5, etc. In an additional example, reference to greater or less than a particular percent, e.g., greater than 25% means 26%, 27%, 28%, 29%, 30%, 31%, . . . etc.; and less than 25% means 24%, 23%, 22%, 19%, 18%, 17%, . . . etc.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes isolation of cells from menstrual blood.

5 ml of menstrual blood was collected from female subjects after informed consent the second day after menstrual blood flow initiated. Collection was performed in a sterile urine cup and then transferred into a 50 ml conical tube (Corning) with 0.2 ml amphotericin B (Sigma-Aldrich, St Louis, Mo.), 0.2 ml penicillin/streptomycin (Sigma 50 ug/nl) and 0.1 ml EDTA-Na2 (Sigma) in a total volume of 40 ml phosphate buffered saline (PBS). Cells were washed by centrifugation at 600 g for 10 minutes, which produced a cell pellet at the bottom of the conical tube. Under sterile conditions supernatant was decanted and the cell pellet was gentle dissociated by tapping until the pellet appeared liquid. The pellet was resuspended in 25 ml of PBS and gently mixed so as to produce a uniform mixture of cells in PBS. In order to purify mononuclear cells, 15 ml of Ficoll-Paque (Fisher Scientific, Portsmouth N.H.) density gradient was added underneath the cell-PBS mixture using a 15 ml pipette. The mixture was subsequently centrifuged for 20 minutes at 900 g. Subsequently the buffy coat was collected and placed into another 50 ml conical tube together with 40 ml of PBS. Cells were centrifuged at 400 g for 10 minutes, after which the supernatant was decanted and the cell pellet was resuspended in 40 ml of PBS and centrifuged again for 10 minutes at 400 g. The cell pellet was subsequently resuspended in 5 ml DMEM medium supplemented with 1% penicillin/streptomycin, 1% amphotericin B, 1% glutamine and 20% FBS (hereafter referred to as completed DMEM). The resuspended cells were mononuclear cells substantially free of erythrocytes and polymorphonuclear leukocytes as assessed by visual morphology microscopically. Viability of the cells was assessed with trypan blue. Of 5 samples tested, all had viability >97%.

### Example 2

This example describes culture of menstrual derived mononuclear cells.

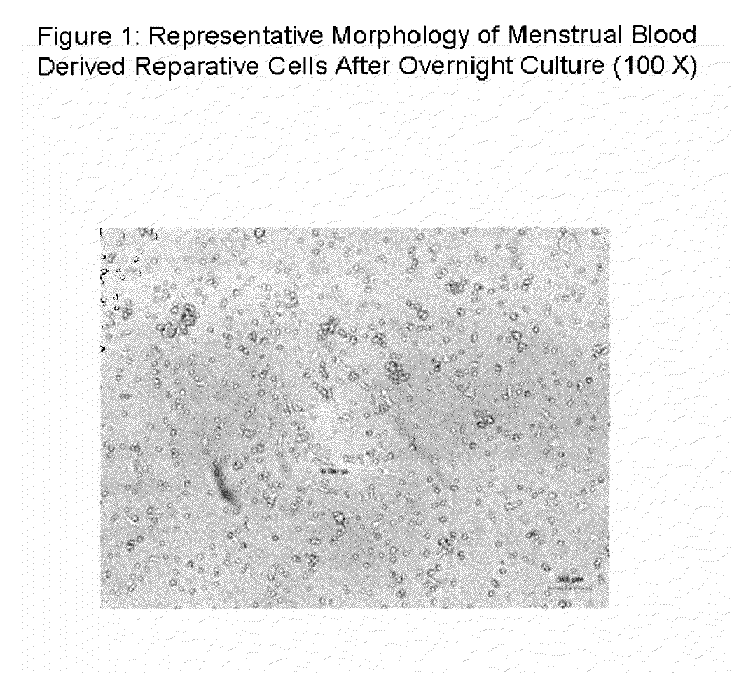

1×10⁶ menstrual blood derived mononuclear cells were placed in a 15 ml sterile Petri dish (Corning, Acton, Mass.) in 10 ml complete DMEM medium. DMEM is a variation of MEM, and contains approximately four times as much of the vitamins and amino acids present in MEM and two to four times as much glucose as MEM. Other tissue culture media may be used such as Roswell Park Memorial Institute Media (RPMI-1640) which is available from Sigma (Product #R6504), Basal Medium Eagle (BME), Ham's, and Minimum Essential Medium Eagle (MEM, or EMEM), which contains amino acids, salts (potassium chloride, magnesium sulfate, sodium chloride, and sodium dihydrogen phosphate), glucose and vitamins (folic acid, nicotinamide, riboflavin, B-12). Cells were cultured overnight at 5% CO2 at 37 degrees Celsius in a fully humidified incubator. After overnight culture, cells were examined under an inverted light microscope. FIG. 1 shows morphology of the cells. The majority of the cells were non-adherent, while a small number could be seen adhering to the Petri dish.

To collect adherent cells, media from the Petri dish was decanted and 10 ml of PBS was added to the Petri dish. The Petri dish was gently rocked back and forth 5 times and PBS was then removed with a pipette, with care being taken not to disrupt adherence cells. This procedure was repeated a second time. Subsequently all PBS is removed and 2 ml of Trypsin-EDTA solution (Sigma Aldrich, St Louis, catalogue #T3924) was added to cover the surface area of the Petri dish. The Petri dish was subsequently placed into an incubator at 37 Celsius for 2 minutes. Cells were then detached by gentle flushing of PBS over the Petri dish. Cells in 10 ml of PBS were centrifuged for 10 minutes at 400 g. The cell pellet was respuspended by tapping gentle against a hard surface 5 times and subsequently complete 5 ml of complete DMEM was added to the resuspended pellet. Cells were counted and 1×10⁵ cells were placed in a T75 flask (Fisher Scientific, Portsmouth N.H.) containing 15 ml of media and cultured in a fully humidified incubator at 37 Celsius, 5% CO2. The cells were then subcultured and passaged twice a week. Passaging involved trypsinization of cells when they reach approximately 75% confluence. After trypsinization and washing 1×10⁵ cells are placed into T75 flasks in 15 ml complete DMEM. Based on these conditions cells are typically passaged 2 times a week.

After 2 week culture cells all assume a fibroblastoid-like morphology and were adherent to the tissue culture flask as seen in FIG. 3.

### Example 3

This example describes culture of menstrual derived membranes.

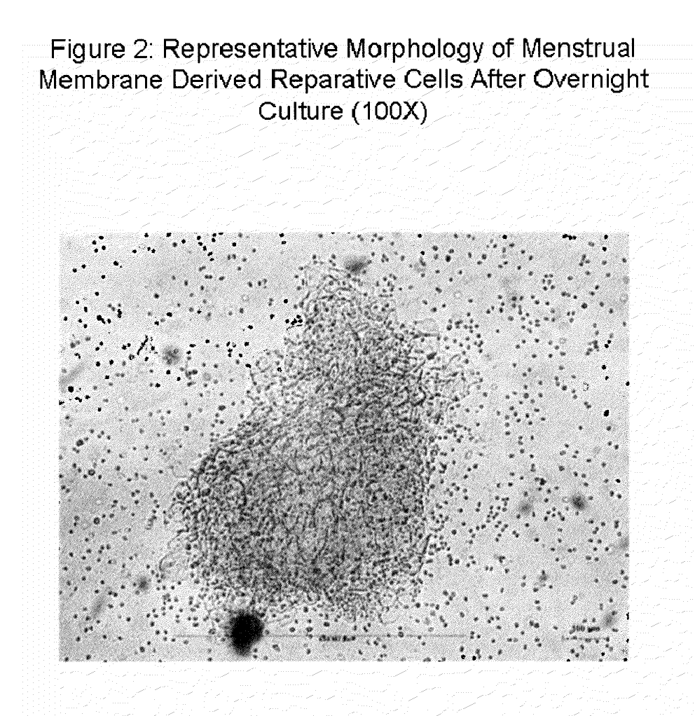

Collection of menstrual blood was performed as described in Example 1. Membranous materials were identified based on microscopic clump-like shapes after menstrual blood was diluted in 40 ml of PBS containing 0.2 ml amphotericin B (Sigma-Aldrich, St Louis, Mo.), 0.2 ml penicillin/streptomycin (Sigma 50 ug/nl) and 0.1 ml EDTA-Na2 (Sigma) in a total volume of 40 ml phosphate buffered saline (PBS). Membranous materials were extracted microscopically using a sterile pipette and placed in complete DMEM media overnight in a fully humidified incubator at 37 Celsius with 5% CO2. An 100× photograph after overnight culture is seen in FIG. 2.

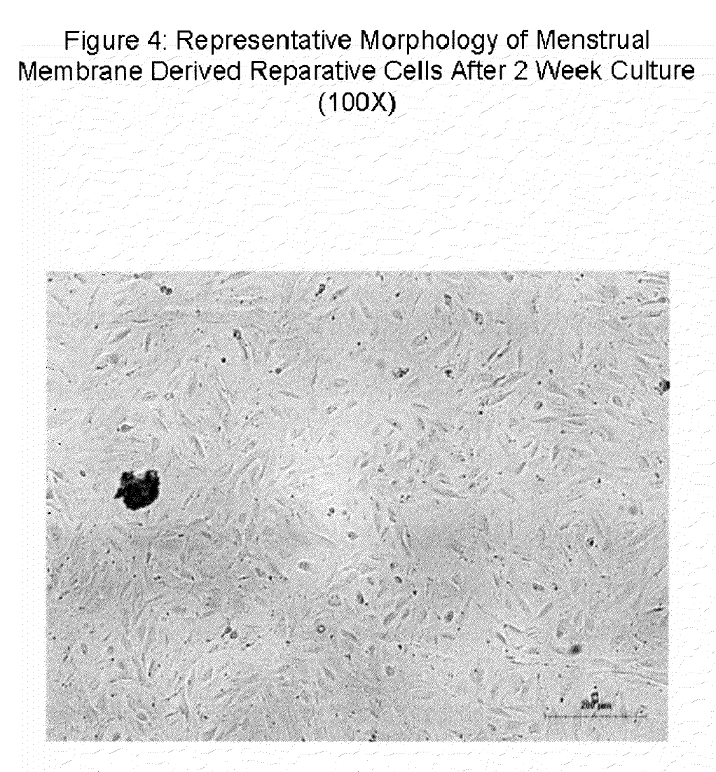

Culture of the menstrual membranes for 48 hours revealed an adherent population attaching to the bottom of the tissue culture plate. Cells were trypsinized as described in Example 2 for menstrual blood derived cells, and passaged similarly. As observed in FIG. 4, the cells exhibited a similar morphology to cells derived from menstrual blood.

### Example 4

This example describes cloning of menstrual blood derived and membrane derived cells.

In order to obtain a homogenous population of cells, adherent mononuclear cells were separated from menstrual blood as described in Example 2 and from menstrual membranes as described in Example 3. Cells were isolated after 2 weeks of culture so as to allow for overgrowth of cells with adherent characteristics.

Cloning was performed by plating cells in flat bottomed 96 well plates at the concentration of approximately 1 cell per well. Wells contained 200 ml of DMEM complete media (Corning, Acton, Mass.). Cells were incubated in a fully humidified incubator at 37 Celsius in a 5% CO2 atmosphere.

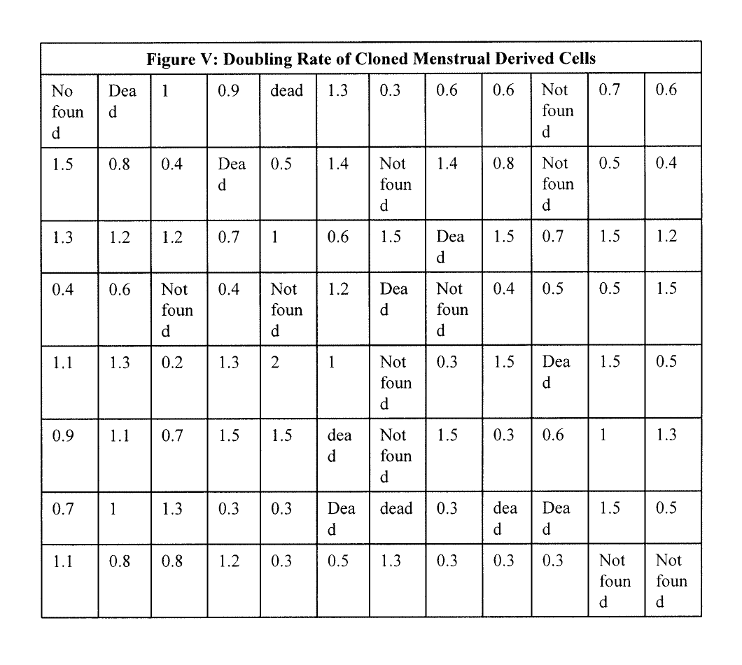

96 wells contained cloned menstrual blood derived cells. An additional 96 well plate contained 96 wells with cloned cells derived from menstrual membranes. Cells possessing a rapid growth characteristic profile, as determined by microscopy were selected for further experiments and generation of cell banks. To determine growth of cells, cells were evaluated every 12 hours for three days. Rate of doubling was calculated on average doublings per day. FIG. 5 (reproduced below) represents a 96 well plate and the doubling rate of cells plated at a 1 cell per well concentration.

Doubling Rate of Cloned Menstrual Derived Cells

| | | | | | | | | | | | |
|---|---|---|---|---|---|---|---|---|---|---|---|
| No | Dead | 1 | 0.9 | dead | 1.3 | 0.3 | 0.6 | 0.6 | Not | 0.7 | 0.6 |
| found | | | | | | | | | found | | |
| 1.5 | 0.8 | 0.4 | Dead | 0.5 | 1.4 | Not | 1.4 | 0.8 | Not | 0.5 | 0.4 |
| | | | | | | found | | | found | | |
| 1.3 | 1.2 | 1.2 | 0.7 | 1 | 0.6 | 1.5 | Dead | 1.5 | 0.7 | 1.5 | 1.2 |
| 0.4 | 0.6 | Not | 0.4 | Not | 1.2 | Dead | Not | 0.4 | 0.5 | 0.5 | 1.5 |
| | | found | | found | | | found | | | | |
| 1.1 | 1.3 | 0.2 | 1.3 | 2 | 1 | Not | 0.3 | 1.5 | Dead | 1.5 | 0.5 |
| | | | | | | found | | | | | |
| 0.9 | 1.1 | 0.7 | 1.5 | 1.5 | dead | Not | 1.5 | 0.3 | 0.6 | 1 | 1.3 |
| | | | | | | found | | | | | |
| 0.7 | 1 | 1.3 | 0.3 | 0.3 | Dead | dead | 0.3 | dead | Dead | 1.5 | 0.5 |
| 1.1 | 0.8 | 0.8 | 1.2 | 0.3 | 0.5 | 1.3 | 0.3 | 0.3 | 0.3 | Not | Not |
| | | | | | | | | | | found | found |

### Example 5

This example describes characterization of cloned menstrual blood stem cells, also referred to as pluripotent stem cells and endometrial regenerative or reparative cells.

Cloned cells from 96-well flat bottomed plates that exhibited doubling rates of approximately 20 hours or shorter (=>1.2 doublings per day), and clones of cells that exhibited doubling rates of approximately 0.5 multiplications per day (one doubling every 48 hours), where identified as described in Example 4. Both clones of the rapidly proliferating cells, as well as the slower proliferating cells were isolated by trypsinization. Isolation was performed by inverting the 96 well plate and tapping the inverted plate against a paper towel under sterile conditions so as to substantially remove tissue culture media (approximately 1-5 microliters of tissue culture media remains per well). 200 ml of PBS was added to the wells by pipette. PBS was subsequently removed by inverting and tapping the 96 well plate against a paper towel. Subsequently 30 microliters of Trypsin-EDTA solution (Sigma Aldrich, St Louis, catalogue #T3924) was added and the 96 well plate was incubated for 2 minutes at 37 Celsius in a fully humidified incubator with 5% CO2. Subsequent to the incubation, 150 microliters of DMEM complete media was pipetted onto each well and the volume of PBS was flushed up and down 5 times to release the cells from adherence to the plastic wells. Cells were placed in a 15 ml sterile Petri dish containing 10 ml of DMEM complete media. Cells were incubated as previously described, for a period of 1 week, with DMEM complete media removed and new DMEM complete media added at 3 days after the incubation. At one week, cells were trypsinized and assessed for marker expression using flow cytometry.

### Example 6

This example describes distinguishing features of rapidly proliferating menstrual blood derived stem cells and slow proliferating cells.

3 clones were selected to represent rapidly proliferating cells (doubling 20 hours or shorter) and slower proliferating cells (doubling 48 hours or longer). Flow cytometry was performed to assess phenotypic differences. Flow cytometry was performed with cells after expansion of clones in a Petri Dish as described in Example 5 (early time point), as well as expansion after approximately 40 doublings (late time point).

Flow cytometry was performed using a Facscalibur (Becton Dickinson, Rockville, Md.). Approximately 50,000 events were quantified. Isotype controls were used for all samples. Cells were stained according to typical laboratory protocols. Specifically, cells, approximately 500,000, were trypsinized as described in Example 10 and admixed with 2 ml of Hanks Buffered Saline Solution (HBSS, Invitrogen, Carlsbad, Calif.) supplemented with 2% bovine serum albumin (Sigma) in 4 ml conical tubes (Invitrogen). Cells were spun in a centrifuge for 600 g for 10 minutes to generate a cell pellet. The supernatant was decanted and the pellet was resuspended by gently tapping. 100 microliters of HBSS with 2% bovine serum albumin (BSA) is added to the tubes and fluorescent (FITC or PE) labeled antibodies are added to cells. Antibodies were added at a concentration of 10 microliters of antibody per tube (concentration of 50 micrograms per ml). Cells with antibodies were incubated on ice for 30 minutes. Subsequently cells are washed 3 times by adding 1 ml of HBSS supplemented with 2% BSA in 4 ml conical tube containing the cells. Cells were spun in a centrifuge for 600 g for 10 minutes to generate a cell pellet. The supernatant was decanted and the pellet was resuspended by gently tapping. Subsequently 1 ml of HBSS and 2% bovine serum albumin is added to the resuspended pellet and the procedure is repeated. At the end of the wash, cells are resuspended in 500 microliters of HBSS and 2% BSA and analyzed by flow cytometery.

Antibodies used were against the following human markers: CD14, CD34, CD38, CD45, CD133, CD9, CD29, CD59, CD73, CD41a, CD44, CD90, and CD105 (BD Pharmingen, Carlsbad, Calif.). Appropriate isotype controls were purchased from the manufacturer and used for all experiments. PE-labelled antibody to STRO-1, HLA-ABC and HLA-DR were purchased from Ancell (Bayport, Minn.), FITC-labeled anti SSEA-4 was purchased from eBioscience (San Diego, Calif.). These antibodies were used to stain the cells in a similar manner as the antibodies to the CD markers mentioned above.

Expression of Nanog, hTERT, and Oct-4 was assessed by intracellular flow cytometry. Cells were washed twice in HBSS with 2% BSA and fixed with 4% Formalin by weight diluted in PBS (Sigma) for 1 hour. Fixing was performed by incubation of the cell pellet with the formalin solution. Subsequently cells were washed twice in 0.5% Tween20 and 0.1% Triton X-100 in PBS (T-PBS). Primary antibodies (Chemicon, anti-Nanog, Abcam anti-hTERT and Oct-4), were added to T-PBS at the concentrations of 1 microgram per ml. Incubation was performed for 30 min. Cells were then washed twice in T-PBS. Corresponding secondary antibodies with fluorescent conjugates PE were subsequently diluted in T-PBS at the concentrations of 1 microgram per ml. Incubation was performed for 20 min and cells were analyzed using flow cytometry.

For flow cytometry analysis, data is presented as positive if staining is found on more than 80% of the cells and level of peak fluorescent intensity is at least 10 fold higher than the level of fluorescent intensity of the isotype control.

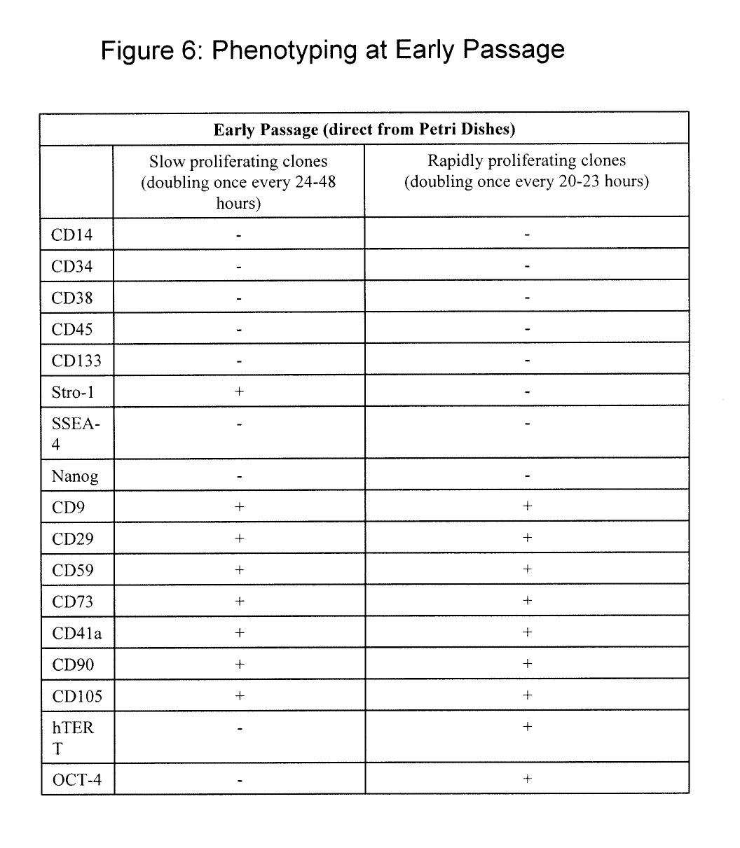

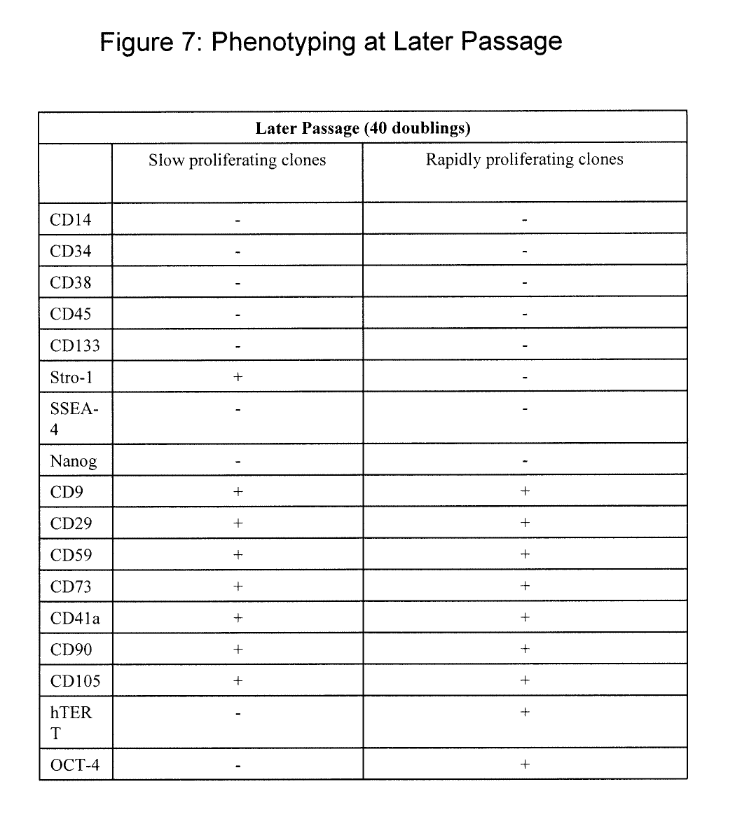

As shown in FIG. 6 a distinct phenotypic difference was seen between cells extracted from slow proliferative versus high proliferative cells. This distinction was maintained after approximately 40 cell doublings. FIG. 7. Specifically, phenotypic differences included the expression of OCT-4 and Telomerase on the rapidly proliferating cells, whereas the slow proliferating cells lacked these markers but expressed STRO-1, which was lacking in rapidly proliferating cells.

Further phenotyping was performed by immunohistochemistry. Cells where stained with the appropriate markers as described above for flow cytometry and observed microscopically under a fluorescent microscope. The staining of the cells was defined as negative if they are not observed under FITC or PE at exposure 1000 and Gain 1.

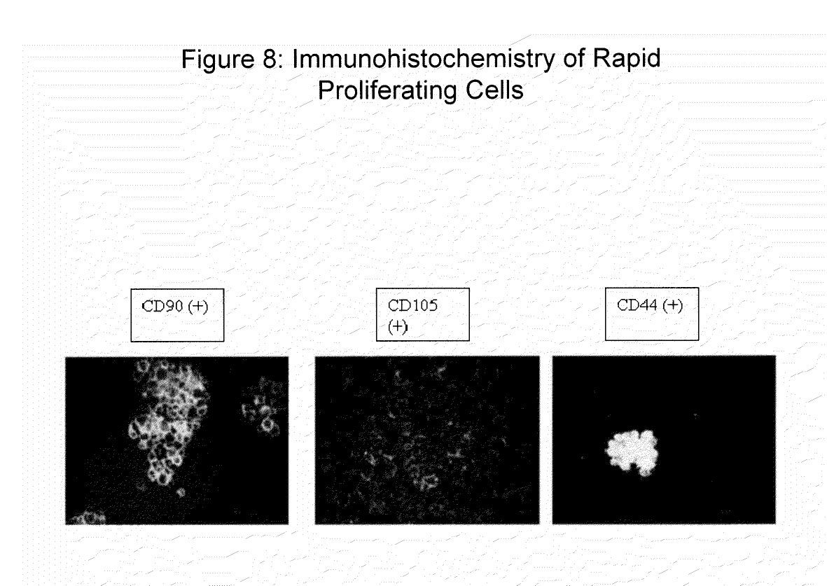

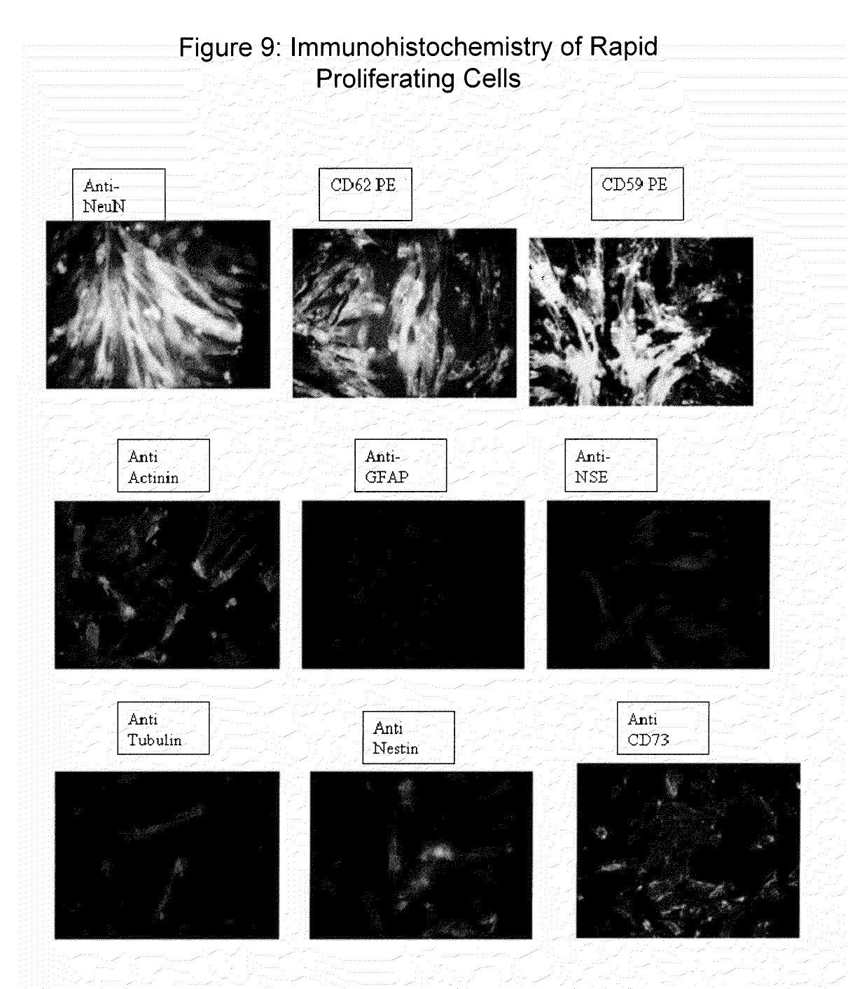

FIGS. 8 and 9 depict positive surface staining of cells derived from rapidly proliferating clones after approximately 40 doublings as positive for CD90, CD 105, CD73, and CD44, thus reconfirming flow cytometry data, as well as positive for NeuN, CD62, CD59, actin, GFAP, NSE, tubulin, and nestin.

### Example 7

This example describes phenotypic characteristics of heterogenous menstrual derived adherent mononuclear cells.

In order to clearly distinguish the need for cloning, data is presented on the phenotypic characteristics of menstrual blood derived mononuclear cells that have not been cloned. Menstrual blood mononuclear cells were harvested as described in Example 1 and cultured under identical conditions with the exception that cells were not cloned. Instead the complete adherent population was maintained in tissue culture and passaged as described in Example 2.

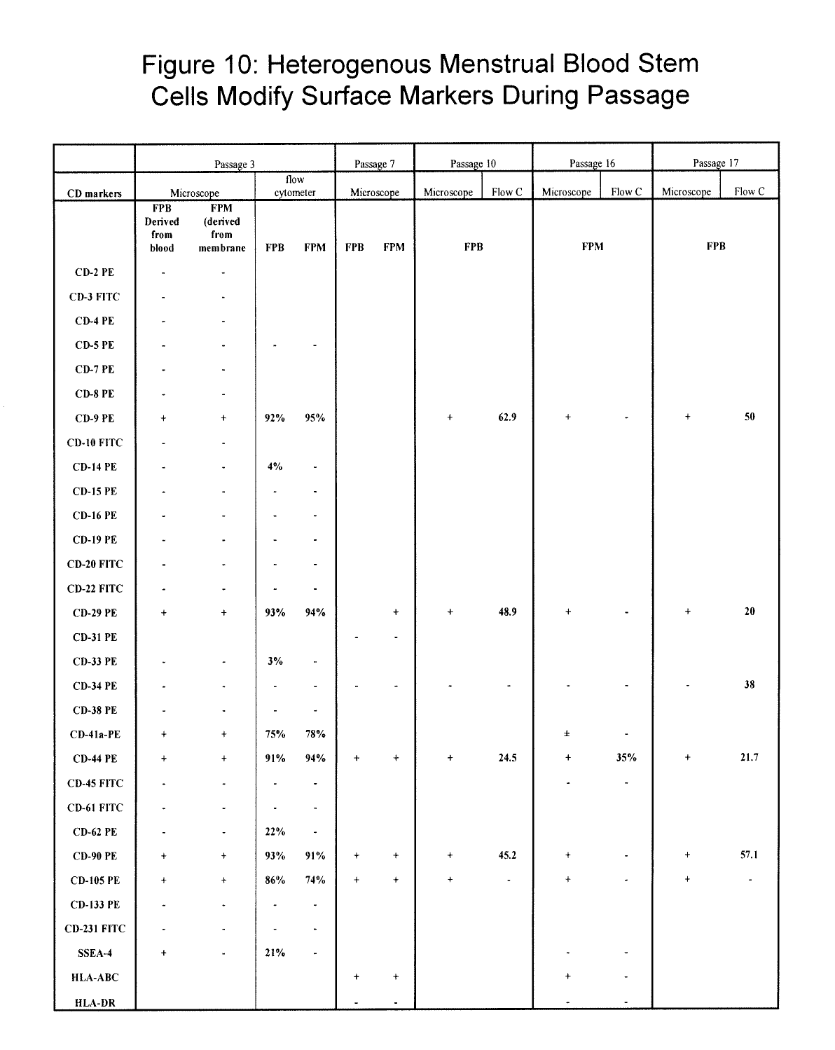

Flow cytometric and microscopic analysis was performed as described in Example 6. As seen in FIG. 10, a gradual decrease in percentage positivity of various cell markers is seen when heterogenous populations of menstrual blood derived mononuclear cells are used.

### Example 8

This example describes karyotypic normality of cloned cells.

High proliferating menstrual blood derived mononuclear cells were passaged for an estimated 70-80 cell doublings and send for karyotypic analysis to NeoDiagnostix, Inc. (Rockville Md.) for karyotypic analysis. Cells were harvested at 70-80% confluency and resuspended in 10 microliters of colcemid per ml of media. Cells were incubated at 37° C. for 3-6 hrs after which cells were resuspended in 0.5 ml medium and mixed with 0.075 M KCl to a volume of 10 ml. After incubation for 10-15 min at 37° C. in a waterbath cells were resuspended to in a total of 10 ml fixative (methonal:acetic acid as 3:1). Staining with DAPI for G-banding was performed by equilibrating the slides in 0.3 M sodium citrate, containing 3 M NaCl for 5 min and subsequent addition of 2 drops of Antifade with DAPI per slide prior to visualization. FIG. 10 depicts karyotypic normality of cells at 70-80 doublings.

### Example 9

This example describes induction of differentiation of pluripotent stem cells.

### Adipogenic Differentiation

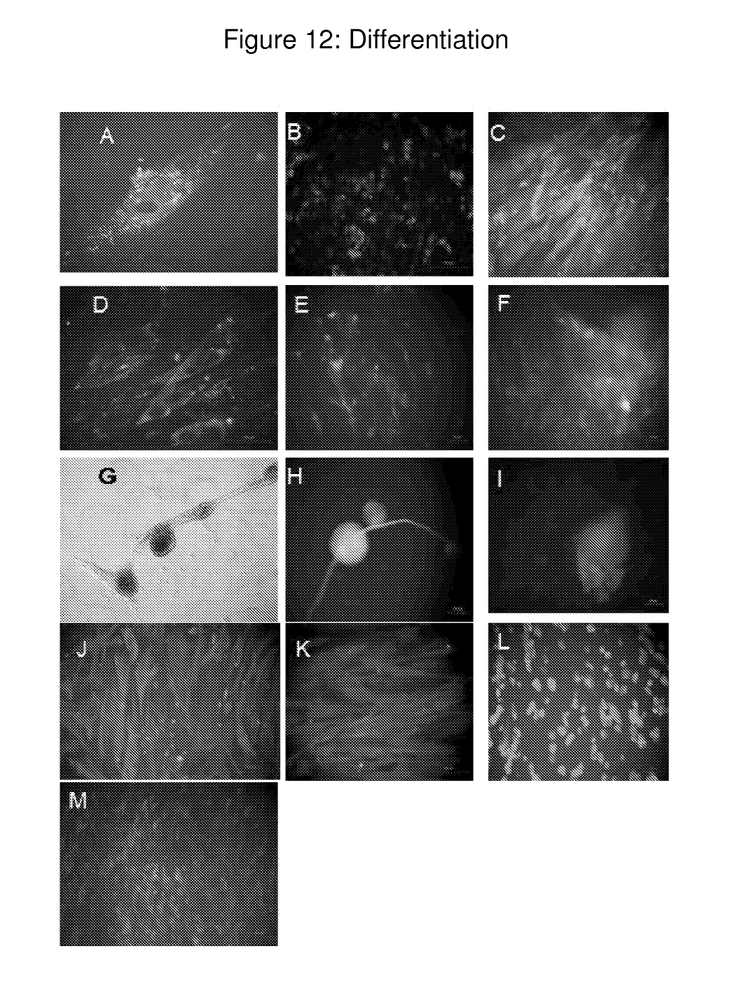

Menstrual mononuclear cells from the high proliferating clones, Example 4, hereafter termed “Endometrial Regenerative Cells” (ERC), at passage 4 (passage 4 cells used for all differentiation experiments), were seeded at a concentration of 4×10[4] cells/ml in an 8 well chamber slide (Lab-Tek, Campbell, Calif.) with 0.5 ml media per well. When the cells reached 100% confluence they were transferred to Adipogenic Induction Media (Cambrex, East Rutherford, N.J., catalogue #PT3004) and cultured for 10 days with media changes every 3-4 days. Control cells were cultured in completed DMEM media. Cells are subsequently stained with AdipoRed (Cambrex) and visualized under fluorescent microscopy. AdipoRed staining was performed by plating differentiated cells in a 6-well plate (Corning) that at a concentration of 30,000 cells/cm2. Cells were plated in 5 ml of PBS with 140 microliters of AdipoRed stain. The stain was dispersed to form a homogeneous mixture by pipetting up and down 3 times a volume of 2 ml. Cells were incubated at room temperature and observed under fluorescent microscopy. As seen in FIG. 12A, differentiated cells assumed an adipocyte-like morphology and stained yellow for lipid vacuoles.

### Osteogenic Differentiation

ERC were seeded at a concentration of 1×10[4] cells/ml in an 8 well chamber slide (Lab-Tek) with 0.5 ml complete DMEM media per well. After the cells where left to adhere overnight, the medium was changed to the Osteogenic Induction media (Cambrex PT3002). Cultures were cultured for 21 days with medium changes every 3-4 days. Control cells were cultured in complete DMEM. Cells were stained with Alizarin Red Solution (ScholAr Chemistry, West Henrietta, N.Y.) and visualized. Staining was performed by removing non-adherent cells and tissue culture media through inversion of the tissue culture plate, followed by addition of the Alizarin Red Solution. The cells were incubated with the solution for a period of 10 minutes and visualized under fluorescent microscopy. As seen in FIG. 12B, cells possessed an osteocyte-like morphology, and stained positive for calcium crystals as noted by the red staining.

### Cardiogenic and Myogenic Differentiation

8 well chamber slides were pre-coated with fibronectin (Sigma #F2006) and ERC were seeded at a concentration of 1.9×10[4] cells/ml. After overnight culture adherent cells were treated with complete DMEM containing 10 μM 5-Azacytidine (Sigma) for 24 hours. Subsequently the cells were cultured for 14 days in Skeletal Muscle Growth Medium (Cambrex CC-3610) supplemented with 100 ng/ml b-FGF (Sigma). Cells were stained with Alpha-Actinin (Abeam) for myocyte and Skeletal Myosin (Abeam, Cambridge Mass.) for skeletal myocyte. Positive staining for Alpha Actinin (FIG. 12C) and Skeletal Myosin (FIG. 12D) was observed.

For the cardiogenic differentiation, cultures are allowed to develop for 40 days with medium changes every 3-4 days and stained with Troponin I (Abeam #AB19615) plus conjugated Goat Anti-mouse (Bethyl #A90-216F). Positive troponin I staining was seen (FIG. 12M). In some experiments cells were grown as hanging drop cultures as described (Pandur et al. What Does it Take to Make a Heart? Biology of the Cell (2005) 97, (197-210)) and incorporated here by reference, in order to visualize beating. Briefly, 30-50 μl of cells were placed on a lid of a petri-dish (Becton Dickinson Falcon #35-3002) and 5-9 ml sterile PBS to bottom of dish to maintain a humidified environment. Beating cells were detected after 5 days.

### Endothelial Differentiation

ERC were seeded at a concentration of 1.9×10[4] cells/ml in an 8 well chamber slide (Lab-Tek) with 0.5 ml complete DMEM per well. After the cells were cultured overnight the media was changed to the Endothelial Induction media (Cambrex CC-3125). Cells were cultured for 21 days with media changes every 3-4 days. Control cells were cultured in complete DMEM. Cells are stained with anti-CD34 and anti-CD62 (Ancell) followed by fluorescently tagged secondary antibody. As seen in FIGS. 12E and 12F, cells were positive for CD34 and CD62 expression. Morphologically, the cells resembled endothelial cells.

### Hepatic/Pancreatic Differentiation

ERC were seeded at a concentration of 2×10[4] cells/ml in an 8 well chamber slide (BD Biosciences #354630) with 0.5 ml complete DMEM media per well. After the cells where incubated to adhere overnight, the medium was changed to the induction medium (Cambrex CC-3198) supplemented with hepatocyte growth factor (40 ng/ml), b-FGF (20 ng/ml), hFGF-4 (20 ng/ml), SCF (40 ng/ml) (all from Sigma). Cultures were maintained for 30 days with media changes every 3-4 days. Cells were stained with antibodies to Albumin (R&D #MAB1455) and insulin and developed plus secondary goat Anti-mouse (Bethyl #A90-216F) and mouse anti-rat (Serotec), respectively. As seen in Figure XIIg, cells possessed a hepatocyte-like morphology. Staining with antibody to albumin revealed expression of this hepatic-specific protein (FIG. 12H). For generation of pancreatic-like cells, addition of glucose at a concentration of 25 mM glucose for the last 7 days of culture. As seen in FIG. 12I, insulin producing cells were detected after the incubation period.

### Neurogenic Differentiation

ERC cells were seeded at a concentration of 1.6×10 [4] cells/ml in an 8 well chamber slide (Lab-Tek) with 0.5 ml complete DMEM. After the cells adhered overnight, the media was changed to the NPMM neural induction media (Cambrex #CC-3209) and supplemented with 1% penicillin/streptomycin, 0.2 mM glutamax (Invitrogen) and hFGA-4 (Sigma F8424, 20 ng/ml). Cultures were cultured in induction or control complete DMEM media for 21 days with media changes every 3-4 days. Cells were stained with GFAP (Sigma) and Nestin (Chemicon), conjugated goat anti-mouse antibody (Bethyl Montgomery, Tex.). FIGS. 12J and 12K depict staining for GFAP and Nestin, respectively.

### Pulmonary Epithelial Differentiation

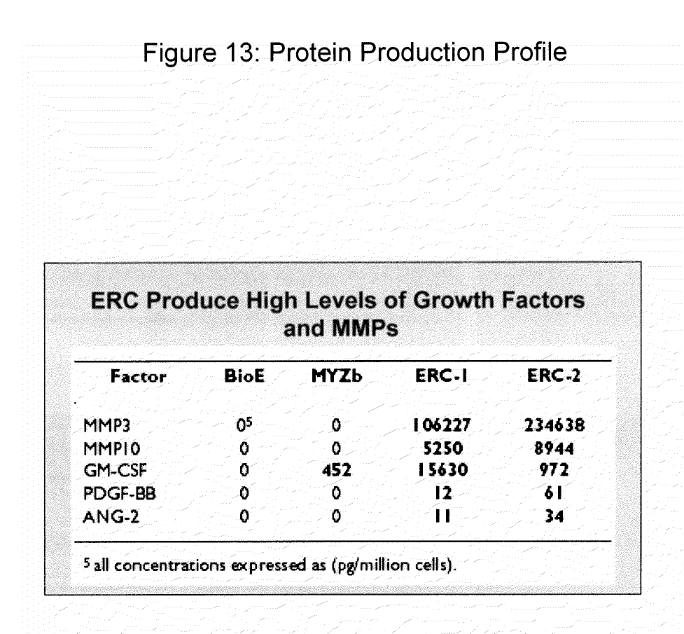

ERC were seeded at a concentration of 2×10[4] cells/ml on 8 well chamber slides (Lab-Tek) with 0.5 ml complete DMEM per well. When the cells reach 100% confluency the media was changed to induction medium (SAGM, Cambrex). Cultures were cultured for 10 days with media changes every 3-4 days. Control cells were cultured in complete DMEM media alone. Cells were stained with ProSP-C (Chemicon) plus conjugated Goat Anti-rabbit (Invitrogen). As depicted in FIG. 13, ProSP-C positive cells were generated after incubation.

### Example 10

This example describes the unique protein production profile of pluripotent stem cells.

Conditioned media was generated from 2 ERC clones (ERC-1 and ERC-2), as well as from control BioE cord blood derived mesenchymal stem cells (St. Paul Minn.) and MYZb cells, an internally-generated cord blood mesenchymal stem cell line. Cells were cultured in T75 flasks for 3 days, with an initial inoculum of 100,000 cells in 15 ml of complete DMEM media. Subsequently, the media were changed to DMEM with 0.2% fetal calf serum. Each flask was rinsed with 10 ml of this media and refilled to 7 ml. After culture for an additional two days, the media was removed and centrifugation at 2000 rpm for 10 minutes was performed to remove cellular debris. Media was frozen at −70° C. for shipping. The cell number in culture was used to calculate the cytokine yield (pg) per million cells. DMEM with 0.2% fetal calf serum (control media) with no cells was sent for the analysis as well. Cytokine release was performed by RayBiotech, Inc (Norcross Ga.) using cytokine array analysis.

As seen in FIG. 13, ERC-1 and -2 produced a substantially higher level of MMP-3 and 10, as well as GM-CSF, PDGF-BB, and Angiopoietin-2 as compared to control cells.

### Example 11

This example describes stimulatory properties of pluripotent stem cell-conditioned media (CM).

Pluripotent stem cells were plated in T-75 flasks at a concentration of 100,000 cells in 15 ml of complete DMEM media. Cells were cultured for 5 days and media was collected. To obtain cell-free conditioned media, the media was centrifuged in 50 ml conical tubes for 40 minutes at 900 g. Supernatant was collected. As control media, complete DMEM media was used.

To evaluate stem cell stimulatory properties, mouse bone marrow cells were extracted from femurs and tibia of 6-8 week old female C57BL/6 mice (Jackson Laboratories, Bar Harbour, Me.). The bone marrow was triturated using an 18 gauge needle and passed through a 70 um nylon mesh cell strainer (Becton Dickinson, Franklin Lakes, N.J.) to make a single cell suspension. Bone marrow mononuclear cells were obtained by by gradient centrifugation over Ficoll-Paque (Amersham Pharmacia Biotech, Uppsala, Sweden). Specifically, cells from femurs and tibia of each mouse were pooled and mixed with complete DMEM media in a total volume of 5 ml. 2 ml of Ficoll was layered underneath. Cells were centrifuged for 40 minutes at 600 g. The buffy coat was collected and washed 3 times in PBS with 3% fetal calf serum.

Bone marrow mononuclear cells were plated at a concentration of 100,000 cells per well in a volume of 100 ml of complete DMEM media. Three concentrations of ERC supernatant were added (20, 40, and 100 microliters of supernatant diluted in non-conditioned complete DMEM media). As a control, conditions media from BioE cells was also used. To generate BioE conditioned media, cells were cultured under identical conditions as pluripotent stem cells.

Bone marrow stem cells were incubated for 48 hours. 1 μCi [³H] thymidine was added to each well for the last 8 hours of culture. Using a cell harvester, the cells were collected onto a glass microfiber filter, and the radioactivity incorporated was measured by a Wallac Betaplate liquid scintillation counter.

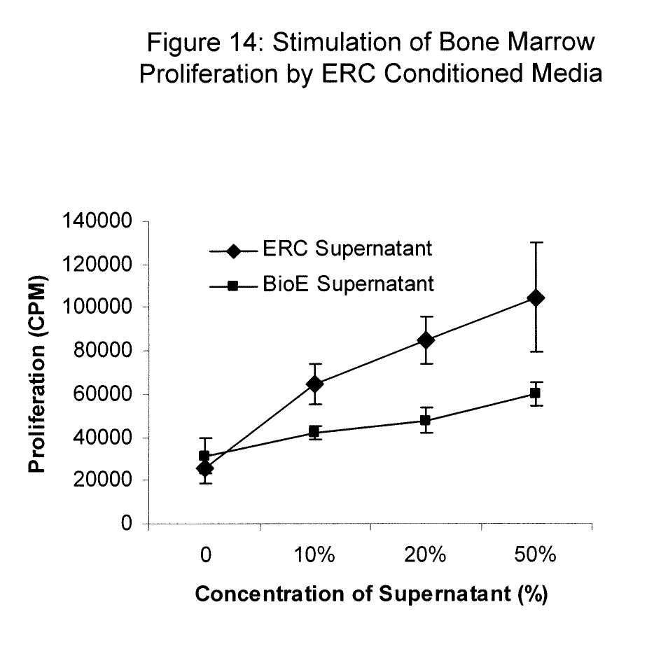

As seen in FIG. 14, a dose dependent increase in bone marrow mononuclear cell proliferation was seen in cultures treated with supernatant from pluripotent stem cells. BioE cell supernatant displayed a trend towards proliferative activity.

### Example 12

This example describes stimulation of human umbilical vein endothelial cell proliferation by pluripotent stem cell conditioned media.

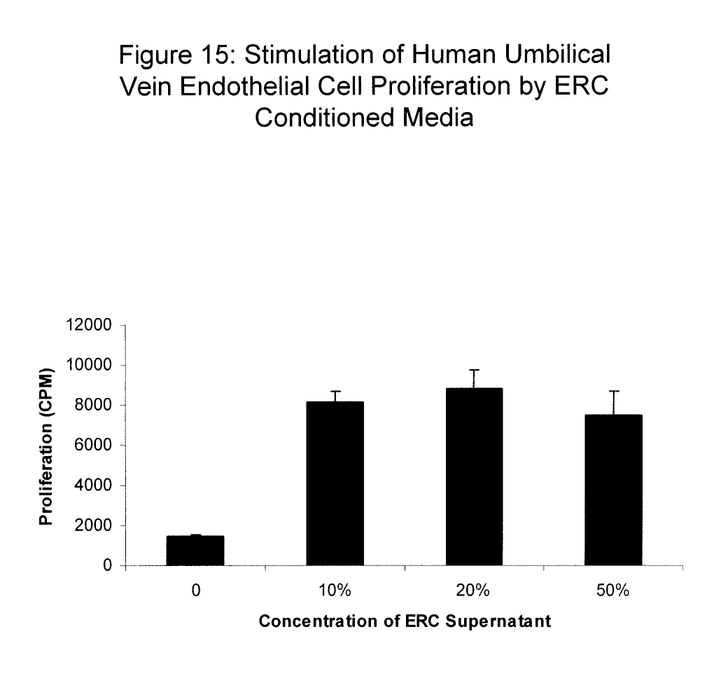

Conditioned media was generated as described in Example 11. In order to assess angiogenic potential, the human umbilical vein endothelial cell (HUVEC) in vitro surrogate assay of angiogenesis was used. HUVEC cells (#CC-2519) and Endothelial Cell Growth Medium (#CC-3024) were purchased from Clonetics (East Rutherford, N.J.). Flat bottomed 96 well plates were coated with 50 micrograms per well of collagen solution and incubated at room temperature for a period of 2 hours. Subsequently wells are washed with 200 microliters of PBS using a pipette. HUVEC cells were diluted in endothelial cell growth medium at a concentration of 50,000 cells per ml. A volume of 100 microliters containing medium and cells was added to each well. An additional 100 ml of complete DMEM (control) was added. To other wells, pluripotent stem cell conditioned media was added to the at concentrations of 20, 40, and 100 microliters of supernatant diluted in non-conditioned complete DMEM media. Cells were cultured for 72 hours at 37 Celsius with 5% carbon dioxide, in a fully humidified environment. For the last 18 hours of culture cells were pulsed with 0.5 μCi 3H-thymidine. In order to quantify proliferation by thymidine incorporation, cells were washed with PBS and 100 microliters of Trypsin EDTA solution was added. Using a cell harvester, the cells were collected onto a glass microfiber filter, and the radioactivity incorporated was measured by a Wallac Betaplate liquid scintillation counter. As seen in FIG. 15, a dose-dependent increase in proliferation of HUVEC cells was seen.

### Example 13

This example describes in vivo stimulation of angiogenesis.

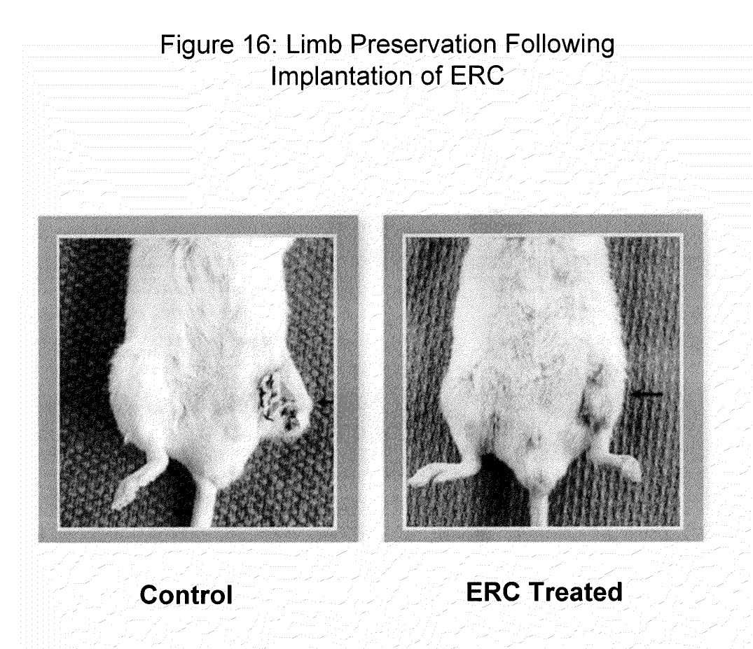

16 BALB/c female mice (6-8 weeks of age, Jackson Labs, Bar Harbor, Me.) underwent unilateral ligation of the femoral artery and its branches (superficial epigastrlc artery) for induction of the limb ischemia. Additionally, ligation of N. peroneus for reproducing a neurotrophic ulcer-like injury was performed. Mice were divided into 2 groups of 8. Immediately after induction of injury, 1 million ERC were injected into the hind-limb muscle below the area of ligation. Cells were also injected on day 0, day 2 and day 4. ERC where injected in a volume of 200 microliters of saline. By day 14, necrosis was observed in legs of 8 control mice. 8 mice treated with ERC had intact limbs, with 2 displaying signs of impeded walking. FIG. 16 depicts a representative control and treated mouse.

### Example 14

This example describes absence of tumorogenic potential of pluripotent stem cells. This example also describes data indicating that pluripotent stem cells can treat cancers, such as melanoma.

16 nude mice (6-8 weeks of age, Jackson Labs, Bar Harbor, Me.) were administered a dose of 0.5 million human ERC cells intravenously. An additional 16 mice received an equivalent number of cells intraperitoneally. Cells were administered in a volume of 200 microlitres. Animals were followed for 4 months, with no sign of tumor or ectopic growth observed at autopsy. Organs assessed included liver, kidney, spleen, heart, intestine, stomach, and peritoneal cavity. General behavior (eating, moving, social interaction) appeared to be unaffected.

18 SKH1 female mice (Charles River Wilmington, Mass.) were treated with 2240 J/m2 of UVB radiation three times a week for 10 weeks to induce skin tumors. Mice were divided into 3 groups of 6 and administered intravenously either 200 microliters of complete DMEM media (Group 1), 500,000 ERC in 200 microliters of complete DMEM (Group 2), or 500,000 human PBMC in 200 microliters of complete DMEM media (Group 3). Administration of cells was performed together with UV irradiation and subsequently on a monthly basis. By day 229 3 mice from Group 2 were alive whereas mice in Groups 1 and 3 succumbed to tumor growth. 1 mouse from Group 2 died on day 245, and the remaining 2 mice from Group 2 where euthanized at day 257 (when the experiment was terminated). Mice appeared to be tumor free at the time of euthanasia.

### Example 15

This example describes clinical safety of pluripotent stem cells.

Clinical preparation of ERC was performed as follows: A healthy female volunteer of 23 years old signed informed consent form for providing menstrual blood sample. The volunteer underwent a standard medical history and examination including evaluation for malignancy, diabetes, leukemia, heart disease. Hematology, biochemistry, and physical examination was uneventful. The patient tested negative for anti-HIV-1, HIV-2, hepatitis B surface antigen, hepatitis B core antibody, VDRL, antibody to trypanosome cruzi, and anti-HTLV-II.

The sample was collected by prefilling a 50 ml tube (Nunc) with 0.5 ml of Antibiotic antimycotic 100× mixture (Gibco) and adding 0.1 ml of EDTA (K3) 15% solution (Cardinal Health, Dublin Ohio). The tube was swirled around 3 times to allow for proper mixing. 5-7 ml of menstrual blood was collected from the healthy volunteer in the sterile tube. Immediately afterwords 40 ml of PBS with 0.4 ml of 100× antibiotic-antimycotic mix was added to the tube. The tube was subsequently centrifuged at 600 g for 10 minutes. The pellet was resuspend in 25 ml of PBS and mixed gently. Subsequently, 15 ml of Ficoll was pipetted under the cell/PBS mix and cells were spun at 900 g for 20 minutes. The buffy coat was collected and transferred into another 50 ml tube to which 40 ml of PBS (with antibiotic-antimycotic mixture). The mixture was centrifuged again for 10 minutes at 400 g. This washing procedure was performed a total of three times. Cells were collected and cultured under conditions for cloning. Clones possessing a rapid proliferative profile (multiplication rate equal to or faster than once every 23 hours) were selected and expanded by culture as described in Example 3 for ERC expansion with the exception that expansion media utilized human umbilical cord serum for the last 3 passages prior to collection for clinical use.

Cells underwent a battery of quality control tests to allow for batch release. These included expression of CD49C, CD90+, CD105+, CD44+, OCT4, (>95% of cells) and lacking expression of CD34, CD45, and CD133 (<5%). Cells were tested for a viability (had to be >97%) by trypan blue, sterility, mycoplasma, and adventitious agents.

Under compassionate use a total of 109 patients have been treated with pluripotent stem cells for a variety of indications under informed consent. The longest follow-up has been more than 12 months with no treatment associated adverse events reported. Cells have been administered intravenously, and/or intramuscularly, and/or intrathecally.

### Example 16

This example describes clinical improvement in diabetic limb function.

3 Patients with Type II diabetes presented. 2 patients suffered from intermittent claudication and one had rest pain. Under informed consent patients where treated with intramuscular administration of 5 million allogeneic pluripotent stem cells (ERC). For administration cells were diluted in saline with 3% autologous serum. Cells were delivered to patients in 0.75 cc aliquots administered 40 times into the gastrocnemius muscle using 3×3 cm grid at a depth of 1.5 cm using 26 gauge needle.

4 weeks-12 weeks after treatment all three patients reported reduction of leg pain and improved walking ability.

### Example 17

This example describes treatment of multiple sclerosis.

5 patients with multiple sclerosis presented with advanced muscle weakness, inability to weak, loss of bowel control, and cognitive impairment. After obtaining informed consent patients were treated with 5 intravenous doses of ERC at a concentration of 3 million cells, as well as 5 intrathecal doses of 3 million ERC. Doses were administered on days 1, 3, 6, 8, and 10. After a period of 4-12 weeks patients report improvement of muscle function, regaining of bowel control and enhanced cognition.

One patient is described in detail: R.H. is a 53 year old male, diagnosed with MS 3 years ago. Patient describes that his symptoms include fatigue, spasticity, spasms, coordination issues and severe neuropathic pain in his right arm, for which he takes a variety of anti inflammatories and narcotics. He received a treatment protocol consisting of 5 intrathecal injections, each one consisting of 6 million ERC's. Treatment protocol included 2 weeks worth of physical therapy. Patient reports that after the second intrathecal injection the neuropathic pain disappeared and that he did not need to take any more analgesics. He reports having an improved feeling of energy and wellness.

### Example 18

This example describes treatment of heart failure.

A patient with ischemic heart failure presents with an expanded left ventricular end systolic volume. The patient felt short of breath upon even mild exertion. After informed consent the patient was treated with 3 million ERC intravenously every other day for a period of 4 total injections. After 12 weeks a reduction in the left ventricular end systolic volume was detected, as well as improved quality of life.

### Example 19

This example describes treatment of a spinal cord injury.

R.O. is a 25 year old male, who had a motorcycle accident 15 months ago, that caused a spinal cord injury at the levels of T5, T6 and T7. The patient had received a treatment protocol consisting in 5 IV injections of ERC's (1 million) 11 months ago. After this treatment protocol, the patient reports having improved movement of his hips that allowed him to transfer to and from his wheelchair more easily. He also mentions having recovered some touch sensation in his right leg.

The patient returned for a follow up treatment. Now he received a treatment protocol consisting of 7 intrathecal injections of ERC's. To this treatment protocol we added physical therapy sessions. In 6 of these intrathecal injections the patient received 6 million ERC's. In one of the injections he received 9 million ERC's (we did this increased dose as a matter of trial). The patient received 7 weeks worth of physical therapy in conjunction of the intrathecal injections. In those 7 weeks the patient was able to stand up with help and walk a few steps using special leg braces and helping supporting himself with his arms on parallel bars. He has regained more sensation to touch in his legs and groin area. Patient still needs to use a catheter to empty his urinary bladder every day, but a urologist he had consulted because of an UTI told us that there are signs that his urinary bladder is changing from being neurogenic to having spasms, which is a sign of reinervation. Most notable is that the patient was able to achieve an erection and ejaculation, which definitely denotes that reinervation is taking place.

### Example 20

This example describes treatment of muscular dystrophy.

A patient Becker's muscular dystrophy presented with general muscle weakness, and difficulty walking. After informed consent the patient was treated with 5 million ERC for a total of 4 treatments administered intravenously spaced at a day apart. Two months after treatment the patient reports an increase in general muscle function and improved walking ability.

### Example 21

This example describes generation of T regulatory cells.

Human peripheral blood mononuclear cells (PBMC) where isolated and cultured at a 1:1 ratio with ERC in round-bottomed 96-well plates that have been precoated with anti-CD3. Cells were incubated for 72 hours. Flow cytometry indicated an increase in CD4+ CD25+T cells. Isolation of T cells from the cultured by magnetic separation and subsequent addition to anti-CD3 anti-CD28 activated T cells resulted in a dose dependent inhibition.

### Example 22

This example describes effect of pluripotent stem cells on mixed lymphocyte reaction (MLR).

Two sets of studies were performed to assess immunological properties of pluripotent stem cells. In the first set, allogeneic PBMC were isolated and cultured at various concentrations with pluripotent stem cells in round bottomed 96 well plates. In order to quantify proliferation of responding allogeneic T cells from the PBMC, pluripotent stem cells were mitotically inactivated by treatment with 10 micrograms/ml of mitomycin C for 2 hours. Subsequently cells were washed with PBS and plated at 10,000, 25,000, and 50,000 cells per well in 96 well plates. Added to the cells were 50,000 allogeneic PBMC. As control stimulator cells, PMBC from a second donor were used. These cells were mitotically inactivated in a manner similar to that used for the pluripotent stem cells. Cells were cultured for 72 hours. For the last 18 hours of culture, cells were pulsed with 0.5 μCi 3H-thymidine. In order to quantify proliferation by thymidine incorporation, cells were harvested and collected onto a glass microfiber filter, and the radioactivity incorporated was measured by a Wallac Betaplate liquid scintillation counter. As shown in FIG. 14, pluripotent stem cells possessed a weak allostimulatory profile as compared to control allogeneic PBMC.

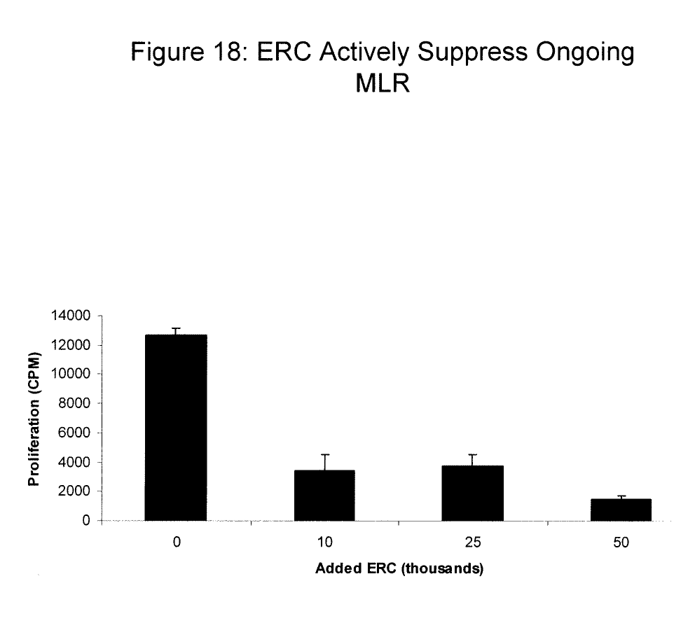

In order to assess active proliferation of ongoing MLR 50,000 mitotically inactivated PBMC (stimulators) were incubated with 50,000 allogeneic PBMC (responders). Mitotically inactivated pluripotent stem cells were added at various concentrations. Cultures were performed using the conditions described above for MLR. An inhibition of proliferation was observed by addition of pluripotent stem cells (FIG. 18).

### Example 23

This example describes modulation of cytokine production by pluripotent stem cells.

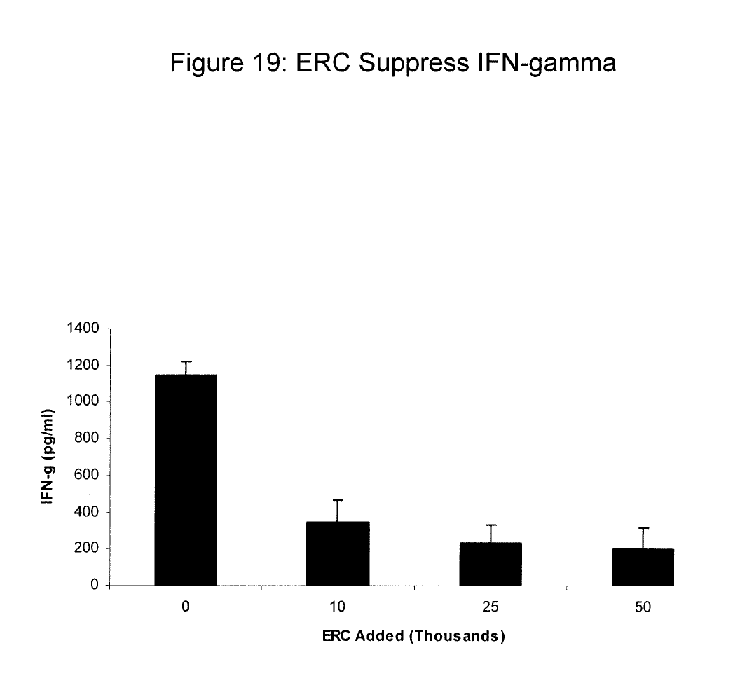

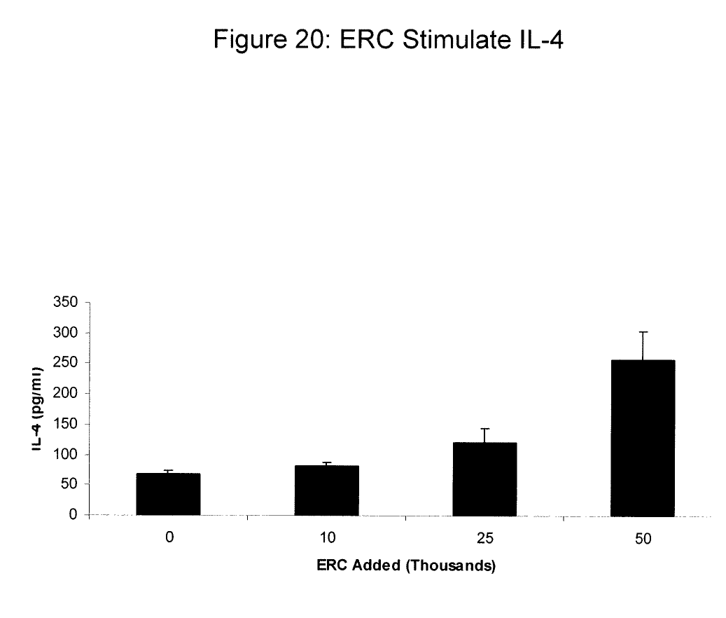

Ongoing MLR was established as described in Example 22 with addition of 3 concentrations of pluripotent stem cells. Instead of assessing proliferation, supernatant was collected from the MLR at 48 hours and assessed for production of interferon gamma (IFN-gamma) (FIG. 19) and interleukin-4 (IL-4) (FIG. 20) by Quantikine Sandwich ELISA (R&D Systems, Minneapolis). As shown in FIGS. 19 and 20, pluripotent stem cells IFN-gamma production and stimulate IL-4 production.

### Example 24

This example describes suppression of TNF-alpha production by pluripotent stem cell conditioned media.

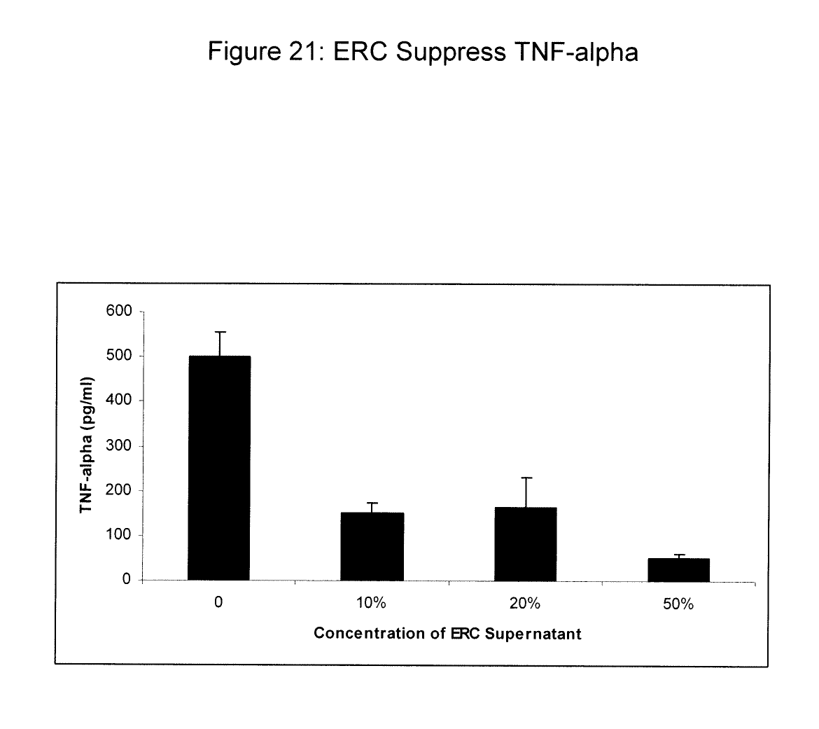

Pluripotent stem cell conditioned media was generated as described in Example 11. Media was added to mouse splenocytes that were activated with 2.5 microliters of lipopolysaccharide (Sigma) in a total volume of 200 microliters. The concentration of splenocytes was 250,000 cells per well. The experiment was performed in 96 well plates. After culture for 48 hours, supernatant was examined for TNF-alpha by ELISA (R&D Systems). FIG. 21 shows inhibition of TNF-alpha production by supernatant from the pluripotent stem cells.

### Example 25

This example describes selective homing of pluripotent stem cells to injured tissue after intravenous injection.

The murine renal ischemia/reperfusion model was used as described by Leemans et al. (J Clin Invest 115:2894). Male BALB/c mice (Jackson Labs) were anesthetized through an intraperitoneal injection of a mixture containing fentanyl citrate 0.08 mg/ml, fluanisone 2.5 mg/ml (VetaPharma Limited) and midazolam 1.25 mg/ml (Roche). Total injection was (80-100 microliters per mouse). After a median abdominal incision, one kidney was removed and the second kidney, the renal artery was clamped for 35 minutes with a microaneuvrysm clamp. Immediately after reperfusion, 5 mice were treated with an intravenous injection of 500,000 ERC that were labeled with CMDil (Chloromethylbenzamido-1,1′-Dioctadecyl-3,3,3′3′-Tetramethylindocarbocyanine Perchlorate: Molecular Probes, USA). 5 control mice that had not been exposed to ischemia reperfusion were also treated with 500,000 CMDil labeled ERC. Labeling was performed by generating a 1 mg/ml solution of CMDil in ethanol and exposing the ERC at a concentration of 8 micromolar for 15 minutes at 37 Celsius. Treatment of cells was performed in the tissue culture flask. Pluripotent stem cells were subsequently trypsinized and injected as described above.

Sections of the kidney were generated by euthanizing mice 48 hours after injection of the pluripotent stem cells and fixing the kidneys in periodate-lysine-paraformaldehyde and embedded in paraffin. 4 micrometer sections where cut and stained with with hematoxylin and eosin. Quantification of labeled cells was performed by a blinded observer. 15 random viewing fields per mouse that were assessed under fluorescent microscopy for CMDil staing (5 mice per group×15 viewing fields=75 in total). Of the total 75 fields observed in the control group (no ischemic injury) 21 cells were counted expressing the CMDil stain. In the mice that underwent ischemia/reperfusion a total of 523 cells were counted.

### Example 26

This example describes a proposed clinical trial for introducing insulin producing cells into one or more subjects in need of insulin producing cells.

Insulin producing cells can be generated from autologous or allogeneic donors. When allogeneic donors are used, matching of the ABO-blood type is still performed.

Administration of insulin producing cells may be performed via the “Edmonton Protocol,” as described, Shapiro, et. al. CMAJ 167:1398 (2002). Patients with type 1 diabetes for more than five years as determined by a stimulated serum C-peptide concentration of less than 0.48 ng per milliliter (0.16 nmol per liter) may be administered immunosuppression immediately before transplantation of pluripotent stem cell-derived insulin producing cells. Immune suppression may consist of sirolimus (Rapamune, Wyeth-Ayerst Canada) administered orally at a loading dose of approximately 0.2 mg per kilogram of body weight, followed by a dose of approximately 0.1 mg per kilogram. Low-dose tacrolimus (Prograf, Fujisawa Canada) may be given orally at an initial dose of 1 mg twice daily, and the dose adjusted to maintain a trough concentration at 12 hours of approximately 3 to 6 ng per milliliter (IMX enzyme immunoassay, Abbott). Daclizumab (Zenapax, Roche Canada) may be given intravenously at a dose of approximately 1 mg per kilogram every 14 days for a total of five doses. After obtaining sufficient numbers of insulin producing cells for transplantation, the patient is given intravenous antibiotics prophylactically (500 mg of vancomycin and 500 mg of imipenem), and oral supplementation with vitamin E (800 IU per day), vitamin B6 (100 mg per day), and vitamin A (25,000 IU per day). Pentamidine (300 mg once a month) is given after transplantation to prevent infection and oral ganciclovir (1 g three times per day) is given for 14 weeks after transplantation to protect against lymphoproliferative disorder.

Prior to administration of pluripotent stem cell-derived insulin producing cells, quality control in terms of cell characteristics, insulin production, karyotypic normality, and insulin secretion in vitro during a glucose challenge is performed. Insulin producing cell preparations are used when they have 4000 islet equivalents per kilogram of the recipient's body weight in a packed-tissue volume of less than 10 ml.

Administration is performed by sedating the patient and a percutaneous transhepatic approach is used to gain access to the portal vein under fluoroscopic guidance. Once access is confirmed, the Seldinger technique is used to place a 5-French Kumpe catheter within the main portal vein. Portal venous pressure is measured at base line and after infusion of the insulin producing cells. The final infusion preparation is suspended in 120 ml of medium 199 that contained 500 U of heparin and 20 percent human albumin and is infused over a period of five minutes. Subsequent to completion of infusion, as the catheter is partially removed, gelatin-sponge (Gelfoam) particles are embolized into the peripheral catheter tract in the liver. Doppler ultrasonography of the portal vein and liver-function tests are performed within 24 hours after transplantation to ensure no damage was performed during implantation procedure.

For use of insulin producing cells in treating allogeneic recipients, in order to avoid the need for immune suppression, which may be detrimental to the patient's long-term health, generated insulin producing cells can be encapsulated so as to avoid immune recognition. Encapsulation may be performed by various means known to one of skill in the art. For example, selectively permeable microcapsules made of Na alginate (AG) and poly-L-ornithine (PLO) may be used to encapsulate cells as described by Calafiore et al (10). Pharmaceutical grade AG powder (Stern Italia, Milano, Italy) can be dissolved in sterile pyrogen-free, deionized water (Italian Pharmacopeia) over the period of 24 to 36 hours in the dark at room temperature; 3% NaCl (Italian Pharmacopeia) is added to adjust the osmolality to physiological levels and the pH was also adjusted to 7.4. The solution is subsequently passaged through methylcellulose and polyester filters (CUNO Italy, Benevento, Italy), dialysis and solution reconstitution, followed by final filtration (0.2 μm) to ensure sterility. The final 1.6% solution may be stored in the dark room at 4° C. to avoid AG depolymerization. Endotoxin levels are measured using the limulus amebocyte lysate method (Cambrex, Brussels, Belgium) or equivalent. Pluripotent stem cells generated insulin producing cells are subsequently encapsulated by centrifuging the cells gently 200 g for 5 minutes in saline with 3% human plasma. The pellet is, approximately several millimeters in size, is then thoroughly mixed with the 1.6% AG solution generated as described above, so as to produce a final homogeneous suspension. The AG/insulin producing cell proportion is adjusted so that one capsule would contain one islet, with fewer than 5% empty capsules. The suspension is extruded through a microdroplet generator, combining air shears with mechanical pressure; the AG droplets are then collected in 1.2% CaCl2 (Sigma Aldrich, Milano, Italy) immediately turning into gel microbeads. The microbeads are sequentially overcoated with PLO and an outer AG layer. The final microcapsule preparations, which should not exceed a final volume of 50 mL, is then incubated for additional 24 hours for sterility and viability checking with ethidium bromide+fluorescein diacetate (Sigma) using fluorescence microscopy.

Encapsulated cells may be administered by a variety of means, for example, by injection into the peritoneal cavity. To perform this, the peritoneal cavity can be imaged using echocardiography and saline is injected to map and detect the capsule deposit area within the peritoneal leaflets. The capsule suspension is diluted in 100 mL of saline (total final volume=150 mL) and delivered with 60-mL plastic syringes having a 16-Guage needle, administered over 10 to 15 minutes.

Other methods of encapsulation of islets are described in U.S. Pat. Nos. 6,911,227, 6,258,870, 5,879,709, 7,018,419, and 6,372,244. Immunoisolatory means of delivering allogeneic cells may also include methods involving the co-implantation of an immune suppressive cell, such as Sertoli cells. Examples of administering potentially immunogenic cells together with immune suppressive cells are described in US Pat. Nos. 5,725,854, 5,849,285, 5,759,534, 5,843,430, 5,958,404, and 6,149,907.

### Example 27

This example describes exemplary methods for producing hepatic-like cells from pluripotent stem cells. This example also describes exemplary animal models of liver failure for analyzing function of the hepatic-like cells derived from pluripotent stem cells.

Pluripotent stem cells are cultured in the presence of extract from damaged liver as described, Ke et. al., Biochem. Biophys. Res. Commun. 367:342 (2008). To stimulate differentiation, pluripotent stem cells are treated with Dkk1 (R&D, USA) at a concentration of 20 ng/ml and Wnt-1 (R&D, USA) at a concentration of 40 ng/ml in complete DMEM. Cells are cultured for 7 days and expression of albumin is used as a marker of hepatic differentiation.

Other methods of inducing differentiation of pluripotent stem cells into the hepatic lineage include in vitro treatment of pluripotent stem cells with 1 micromolar 5-azacytidine (5-aza) for 24 hours and subsequent culture in 20 ng/ml hepatocyte growth factor (HGF), 20 ng/ml oncostatin M (OSM), and 10 ng/ml fibroblast growth factor 2 (FGF2) for 3 weeks using a method described for pluripotent cord blood cells,Yoshida, et. al. Am J Physiol Gastrointest. Liver Physiol. 293:G1089 (2007). Generated hepatocytes or hepatocyte-like cells may be analyzed for expression of proteins such as albumin, CCAAT enhancer-binding protein, and cytochrome p450 1A1/2 in vitro. Additionally, periodic acid-Schiff staining and morphology may be used to determine the similarity between pluripotent stem cells-differentiated hepatocytes and naturally obtained hepatocytes.

Animal models of liver failure may be used to assess efficacy of in vitro generated hepatocytes. For example, the carbon tetrachloride model provides a good standard for assessment of toxin-induced hepatic injury Kobayashi, et. al., Hepatology 31:851 (2000), and partial hepatectomy models allow assessment of endogenous regenerative activity Michalopoulos, G. K. 2007. Liver regeneration. J Cell Physiol 213:286-300. For clinical assessment of pluripotent stem cell-generated hepatocyte-like cells, protocols for hepatocyte transplantation may be used. Such protocols are described in Fox et. al., N Engl J Med 338:1422 (1998).

### Example 28

This example describes pluripotent stem cells for treatment of critical limb ischemia (CLI). An exemplary clinical trial for treatment of CLI is also described.

CLI is caused by arterial occlusion affecting the limbs, usually caused by atherosclerosis or in a smaller number of patients by thromboangiitis obliterans (Buerger's Disease), or arteritis. This condition is a major cause of morbidity and mortality: Approximately 20-45% of patients require amputation, and 1-year mortality is estimated to be as high as 45% in patients who have undergone amputation Dormandy, J. A., and Rutherford, R. B., J. Vasc. Surg. 31:S1 (2000). Some authors have went so far as to compared the quality of life of patients with CLI to terminal cancer patients.

Current treatment options for CLI patients are limited. According to the Inter-Society Consensus for the Management of Peripheral Arterial Disease (TASC II) treatment for CLI should be focused on revasularization using surgical or percutanous means Norgren, et. al., Eur. J. Vasc. Endovasc. Surg. 33:S1 (2007). Unfortunately less than half of the patients may undergo these procedures, and efficacy is limited due to high levels of restenosis and need for re-surgery. Non-surgical options for CLI are limited to medical therapy, which offers limited or no benefit.

Patients with critical limb ischemia (CLI) can be treated by injection (e.g., intramuscular) of allogeneic pluripotent stem cells. The pluripotent stem cells for treatment of CLI may be administered intramuscularly following protocols used for other cell types in the treatment of CLI. Protocols of administration have been described by Lenk, et al., Eur. Heart J. 26:1903 (2005); Huang, et. al., Diabetes Care 28:2155 (2005); Nizankowski, et. al., Kardiol. Pol. 63:351 (2005); Kajiguchi, et. al., Circ. J 71:196 (2007); Lachmann, N. and Nikol, S., Vasa. 36:241 (2007).

An exemplary description of a clinical trial is as follows: 22 patient dose-escalating (10 million, 20 million; and 40 million cells in total) study in a highly defined patient population utilizing GCP-grade monitoring and follow-up. The study will be used for publication and subsequent submission to the FDA to support clinical trials in the USA.

On Visit 1: Patients will be entered into the study upon meeting the inclusion/exclusion criteria (see inclusion/exclusion criteria section below). On Visit 2: Baseline Assessments will be performed (Baseline assessments must be taken within 1 week of study inclusion), this includes the following: a) MRI of affected limb: b) Ankle Brachial Index (ABI) assessement; c) assessment of Pain free walking distance; d) measurement of Transcutaneous oxygen (TcPO2); e) Peripheral nerve conduction assessment; f) VAS pain assessment; g) Quality of life questionnaire; h) Wound healing: diameter and depth of ulcers will be measured and photographed. Visit 3: Pluripotent stem cell administration will be performed. This includes: a) Treatment with local anesthesia and topical disinfection; b) injection of ERC in 0.75 cc aliquots administered 40 times into gastrocnemius muscle using 3×3 cm grid at a depth of 1.5 cm using 26 gauge needle. 3 dose are used depending on the group the patients are entered into. Visit 4: Week 1 Follow-up. Assessed will be: a) ABI; b) VAS pain assessment; c) Pain free walking distance; d) Transcutaneous oxygen (TcPO2); e) Peripheral nerve conduction assessment; f) Quality of life questionnaire; g) Safety & Concomitant Medication Evaluation; h) Serum chemistry; and i) CBC. Visit 5: Week 4 Follow-up. Assessed will be; a) ABI; b) VAS pain assessment; c) Pain free walking distance; d) Transcutaneous oxygen (TcPO2); e) Peripheral nerve conduction assessment; f) Quality of life questionnaire; g) Safety & Concomitant Medication Evaluation; h) 12 lead EKG; i) Serum chemistry; and j) CBC. Visit 6: Week 8 Follow-up. Assessed will be: a) ABI; b) VAS pain assessment; c) Pain free walking distance; d) Transcutaneous oxygen (TcPO2); e) Peripheral nerve conduction assessment; f) Quality of life questionnaire; g) Safety & Concomitant Medication Evaluation; h) 12 lead EKG; i) Serum chemistry and j) CBC. Visit 7: Week 12 Follow-up. Assessed with be: a) ABI; b) VAS pain assessment; c) Pain free walking distance; d) Transcutaneous oxygen (TcPO2); e) MRI; and f) Peripheral nerve conduction assessment.

Detailed procedures used for the study are described below.

Physical examination: A complete history and physical examination with an emphasis on relief of pain, presence of infection or other physical indications (dependent rubor) that the ischemic limb is not improving will be performed at baseline, post-operative day 1 and post-operative weeks 1, 4, 8, and 12. During this time period all adverse and serious adverse events will be recorded.

Rest Pain assessment. Pain assessment will be evaluated with a self-administered visual analog scale at baseline and at weeks 2, 4, 6, 8, and 12. Changes from baseline will be used to chart patient's ongoing perception of pain and will be compared at each time point.

Ankle-Brachial pressure measurements (ABI), Absolute toe pressure and Toe-Brachial Index (TBI). Blood pressure cuffs will be placed on both upper arms and ankles and inflated to approximately 30 mmHg above the systolic blood pressure. As the cuff is deflated the Doppler flow signal will be used to detect the reappearing signal at the right brachial artery, right posterior tibial artery, and right dorsalis pedal artery in sequence. A toe pressure recording will be obtained at the first toe digital artery. This process will then be repeated on the left side at the same sites. The measurements will then be expressed as a ratio or index of the pressures recorded: tibial and pedal pressures/arm pressure (Ankle-Brachial Index) and toe pressure/arm pressure (Toe-Brachial Index). The first toe digital pressure will be recorded without cuff occlusion as the absolute toe pressure. Room temperature will be kept as close to possible to 25° C. and the index measurements will be recorded at rest and when physically feasible after exercise. For patient inclusion and follow-up examination, the following measurements will be used: ankle systolic pressure in the dorsalis pedis and posterior tibial arteries (at rest), brachial blood pressure (systolic at rest), great toe pressure (systolic at rest). ABI and TBI will be obtained at baseline and at weeks 4, 8, and 12 after treatment. In those patients in whom the ankle or toe pressure measurements cannot be obtained due to calcification pulse volume recordings (PVR) will be obtained on the dorsum of the foot. The PVR measurement will be used for patient inclusion criteria only and will not be followed for a change with treatment.

Transcutaneous oximetry. Transcutaneous oximetry (TcPO2) measurements will be obtained at baseline and at weeks 4, 8, and 12 after treatment. The room temperature will be maintained at 25° C. with the patient supine and at rest for a minimum of 30 minutes. Measurements will be recorded after 30 minutes of continuous monitoring. The lowest measurement on the foot will be used as baseline and an indelible marking pen will be used to minimize the variation in follow-up studies. Changes from baseline will be recorded for each patient and compared for each time point. A chest wall measurement will be used to assess reliability of the test over time.

Ulcer Status. The statuses of the two worst ulcers in the index limb will be evaluated at baseline and followed at weeks 2, 4, 8, and 12 post treatment visits. Only index ulcers will be evaluated for change however any newly formed ulcers will be noted during the follow-up period. Index ulcers will grade as to location, size, depth, and type.

Magnetic Resonance Imaging (MRI). Magnetic resonance imaging will be used in this study to visualize newly developed collateral vessels in the index leg. MRI can assess the overall muscle mass and degree of fibrosis which are indirect indices or perfusion status of the extremity. At baseline, imaging of the calf muscles will be performed. This imaging will then be followed by a velocity flow mapping sequence (at the level of the iliac arteries) to evaluate total blood flow to the index and opposite leg. Calf perfusion will then be measured during pharmacologic stress with adenosine using a fast gradient-echo, first pass perfusion sequence with administration of i.v. gadolinium contrast. Velocity flow measurements will be repeated with adenosine on board. Fifteen minutes after adenosine is terminated, calf perfusion measurements will be repeated at rest. This sequence will produce perfusion and flow velocity measurements at rest and with adenosine. Five minutes after the velocity-perfusion study, delayed contrast-enhanced imaging of the calf muscles using a segmented inversion-recovery MR sequence will be performed in order to identify and quantify areas of tissue fibrosis and scarring. Exclusions to this study include but are not limited to a cardiac pacemaker, implanted cardiac defibrillator, aneurysm clips, carotid artery stents, neurostimulators, insulin or similar infusion pump, cochlear, otologic, or ear implants and for these patients arteriography will be the only study employed to visualize flow to the index leg. Perfusion and flow velocity measurements will be compared at 12 weeks after treatment to baseline.

For entry into the trial subjects must have baseline evaluations performed within 7 days prior to the first dose of cells and must meet all inclusion and exclusion criteria. Results of all baseline evaluations, which assure that all inclusion and exclusion criteria have been satisfied, must be reviewed by the Principal Investigator or his/her designee prior to enrollment of that subject. The subject must be informed about all aspects of the study and written informed consent must be obtained from the subject prior to study procedures. The inclusion criteria will include: a) Non-pregnant patients 18 years of age or greater with unreconstructable grade II category 4 ischemia (ischemic rest pain) and grade III category 5 ischemia (ulceration or tissue necrosis); b) Unreconstructable arterial disease will be determined by an interventional radiologist and vascular surgeon who are not participating in the study. Unreconstructable arterial disease is defined by atherocclusive lesions with the arterial tree of the limb, that due to extent or morphology are not amenable to surgical bypass or PTCA and stenting; c) Objective evidence of severe peripheral arterial disease will include an ankle brachial index (ABI) of less than 0.5, a resting toe brachial index (TBI) of less than 0.4, or metatarsal pulse volume recording (PVR) that is flat or barely pulsatile in the diseased limb on 2 consecutive examinations performed at least 1 week apart; and d) No history of malignant disease, no suspicious findings on chest x-ray, mammography, Papanicolaou smear, and a normal prostate specific antigen.

The exclusion criteria will include: a) Patients with evidence of proliferative retinopathy on ophthalmologic examination; b) Patients with poorly controlled diabetes mellitus (HbAlC>6.5%) will be excluded from the study; c) Patients with renal insufficiency (Creatinine>2.5) or failure; d) Patients with congestive heart failure (Ejection Fraction<30%); e) Infection of the involved extremity manifest by fever, purulence, cellulitis and an elevated white blood cell count and f) Pregnant women or cognitively impaired adults.

Pluripotent stem cells can be used for clinical treatment as a stand-alone agent or in combination with other cells or agents. For example, pluripotent stem cells may be used in conjunction with other angiogenesis therapies. Without being limited to any particular theory, pluripotent stem cells may synergize with other agents or cells that are pro-angiogenic, based on the ability of pluripotent stem cells to secrete high levels of matrix metalloproteases. It has been reported that local production of chemoattractant factors occurs when stem cells are administered into an ischemia muscle, Kajiguchi, et. al., Circ. J 71:196 (2007). Given the high amount of angiogenic factors produced by pluripotent stem cells, mobilization of endogenous bone marrow stem cells with agents such as G-CSF, concurrent with administration of pluripotent stem cells in the ischemic muscle. Administration of G-CSF may be performed concurrently with intramuscular injection of pluripotent stem cells, or may be performed near the timepoint associated with maximal mobilization of CD34 cells induced by the pluripotent stem cells administration. The timepoint may be determined empirically, or may be based on previously published data. For example, it was reported that maximal CD34 mobilization subsequent to administration of bone marrow cells intramuscularly occurs around day 30, Kajiguchi, et. al., Circ. J 71:196 (2007). Accordingly, G-CSF can be administered prior to day 30, at concentrations sufficient to evoke endogenous CD34 mobilization. Particular G-CSF doses administered can be at a concentration of approximately 60 migrograms per day be subcutaneous injection for 5 days.

Administration may be performed, for example, starting on day 25 subsequent to intramuscular injection of pluripotent stem cells. Heparin (e.g., approximate doses of 10,000 units per day) may also be concurrently administered so as to avoid the possibility of causing embolism due to high systemic leukocyte counts caused by the G-CSF injection. This is important in CLI patients with NIDDM who are already at a higher risk of embolisms in comparison to the general population. Anticoagulation methods are known in the art and may utilize agents besides heparin.

### Example 29

This example describes pluripotent stem cells administered together with cord blood expanded CD34 stem cells to obtain synergy of regenerative activity.

To generate cord blood expanded CD34 cells umbilical cord blood is purified according to routine methods (e.g., Rubinstein, et al. Proc Natl Acad Sci USA 92:10119). Briefly, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from healthy delivery from a mother tested for viral and bacterial infections according to international donor standards. Cord blood is allowed to drain by gravity so as to drip into the blood bag. The placenta is placed in a plastic-lined, absorbent cotton pad suspended from a specially constructed support frame in order to allow collection and reduce the contamination with maternal blood and other secretions, The 63 ml of CPD A used in the standard blood transfusion bag, calculated for 450 ml of blood, is reduced to 23 ml by draining 40 ml into a graduated cylinder just prior to collection. This volume of anticoagulant matches better the cord volumes usually retrieved (<170 m1). An aliquot of the blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte rich supernatant is then separated by centrifuging the cord blood hydroxyethyl starch mixture in the original collection blood bag (50×g for 5 min at 10° C.). The leukocyte-rich supernatant is expressed from the bag into a 150-ml Plasma Transfer bag (Baxter Health Care) and centrifuged (400×g for 10 min) to sediment the cells. Surplus supernatant plasma is transferred into a second plasma Transfer bag without severing the connecting tube. Finally, the sedimented leukocytes are resuspended in supernatant plasma to a total volume of 20 ml.

Approximately 5×10⁸-7×10⁹ nucleated cells are obtained per cord. Cells are cryopreserved according to the method as described by Rubinstein et al. for subsequent cellular therapy. CD34 cells are expanded by culture. CD34+ cells are purified from the mononuclear cell fraction by immuno-magnetic separation using the Magnetic Activated Cell Sorting (MACS) CD34+ Progenitor Cell Isolation Kit (Miltenyi-Biotec, Auburn, Calif.) according to manufacturer's recommendations. The purity of the CD34+ cells obtained ranges between 95% and 98%, based on Flow Cytometry evaluation (FACScan flow cytometer, Becton-Dickinson, Immunofluorometry systems, Mountain View, Calif). Cells are plated at a concentration of 10.sup.4 cells/ml in a final volume of 0.5 ml in 24 well culture plates (Falcon; Becton Dickinson Biosciences) in DMEM supplemented with the cytokine cocktail of: 20 ng/ml IL-3, 250 ng/ml IL-6, 10 ng/ml SCF, 250 ng/ml TPO and 100 ng/ml flt-3L and a 50% mixture of LPCM. LPCM is generated by obtaining a fresh human placenta from vaginal delivery and placing it in a sterile plastic container. The placenta is rinsed with an anticoagulant solution comprising phosphate buffered saline (Gibco-Invitrogen, Grand Island, N.Y.), containing a 1:1000 concentration of heparin (1% w/w) (American Pharmaceutical Partners, Schaumburg, Ill.). The placenta is then covered with a DMEM media (Gibco) in a sterile container such that the entirety of the placenta is submerged in said media, and incubated at 37° C. in a humidified 5% CO₂ incubator for 24 hours. At the end of the 24 hours, the live placenta conditioned medium (LPCM) is isolated from the container and sterile-filtered using a commercially available sterile 0.2 micron filter (VWR). Cells are expanded, checked for purity using CD34-specific flow cytometry and immunologically matched to recipients using a mixed lymphocyte reaction. Cells eliciting a low level of allostimulatory activity to recipient lymphocytes are selected for transplantation and use together with pluripotent stem cells.

### Example 30

This example describes pluripotent stem cells (or progeny) to be administered to treat an insulin resistant subject by improving vascular function.

Patients suffering from insulin resistance, having a state of NIDDM, can betreated by administration (e.g., intramuscular) of pluripotent stem cells. About 70-80% of post-prandial glucose is metabolized by skeletal muscle, DeFronzo, R. A., Diabetes 37:667 (1988). In many patients with NIDDM, profound atherosclerotic deposits inhibit circulation of the extremities. Without being bound by any particular theory, inhibition of circulation may be occurring at vessels such as the femoral artery, the popliteal artery and/or the tibial arteries. Additionally inhibition of circulation may be occurring at the level of capillaries feeding various muscles. Impaired circulation is may also be due to inhibited vasodilatory mechanisms, Cheetham, et. al., Clin. Sci. (Loud) 100:13 (2001). Due to inhibited circulation and vasodilatory responses, insulin activation of GLUT4 membrane localization and general insulin responsiveness is impaired.

Accordingly, the ability of muscles to respond to insulin is improved by administration of pluripotent stem cells, which may function by restoring or repairing endothelial function, as well as inducing, increasing, stimulating, promoting, enhancing or augmenting angiogenesis. Pluripotent stem cells useful for this purpose may be autologous, endogenous, or allogeneic origin.

### Example 31

This example describes pluripotent stem cells (or progeny) administered to an inflammatory or autoimmune disorder in a subject.

Inflammatory and autoimmune disorders and diseases may be treated with pluripotent stem cells. A non-limiting example is ulcerative colitis is treated.

To assess efficacy, a double blind, randomized study may be performed. To allow for regulatory approval a population of 110 patients is enrolled to allow for proper statistical significance. Patients are enrolled and randomized into either the placebo or treatment group. Eligible patients are assessed for baseline (pre-treatment) clinical values and treated with daily placebo cell therapy administration, or pluripotent stem cells. Patients are allowed to continue taking current treatment, however medical need for escalation of current (non experimental) treatment leads to exclusion of the patient from the study.

Evaluation occurs at Weeks 2, 4, 8, and 10 in the form of the ulcerative colitis disease activity index (score 0-12). Patients undergo endoscopy at Baseline, and Week 8 for assessment of inflammation and pathology using the system defined by Geboes. Other observations will include the number of bowel movements, visible blood in stool, abdominal pain, body temperature, pulse rate, haemoglobin, erythrocyte sedimentation rate (ESR), and serum C reactive protein (CRP) level.

Inclusion Criteria:
1. Age 18 years old or greater.
2. Diagnosis of ulcerative colitis for at least 4 months based on endoscopic appearance or radiographic distribution of disease and corroborated with histopathology (especially the absence of granulomata).
3. Ulcerative colitis DAI greater than or equal to 4 and less than or equal to 9.
4. Active ulcerative colitis that is poorly controlled despite concurrent treatment with oral corticosteroids and/or immunosuppressants as defined: -Stable (±5 mg) corticosteroid dose (prednisone<=20 mg/day or equivalent) for at least 14 days prior to Baseline, or maintenance corticosteroid dose (prednisone<=10 mg/day and <20 mg/day or equivalent) for at least 40 days prior to Baseline -At least a 90 day course of azathioprine or 6-MP prior to Baseline, with a dose of azathioprine<=1.5 mg/kg/day or 6-MP<=1 mg/kg/day (rounded to the nearest available tablet formulation), or a dose that is the highest tolerated by the subject (e.g., due to leukopenia, elevated liver enzymes, nausea) during that time. Subject must be on a stable dose for at least 28 days prior to Baseline.

Exclusion Criteria
1. History of subtotal colectomy with ileorectostomy or colectomy with ileoanal pouch, Koch pouch, or ileostomy for ulcerative olitis or is planning bowel surgery
2. Received previous treatment with rapamycin or previous participation in an rapamycin clinical study
3. Current diagnosis of fulminant colitis and/or toxic megacolon
4. Subject with disease limited to the rectum (ulcerative proctitis)
5. Current diagnosis of indeterminate colitis
6. Current diagnosis and/or history of Crohn's disease
7. Currently receiving total parenteral nutrition (TPN)
   A total of approximately 50×10⁶ pluripotent stem cells are concentrated in injectable saline with 3% autologous serum and injected intravenously. Patients in the placebo group are injected with saline and 3% autologous serum in order not to bias the patients based on color of the solution being injected. Injections are administered weekly for a period of 4 weeks.

The primary end point of the trial is a positive response as determined by a decrease in the DAI by greater than or equal to 3 points at week 8 that was not accompanied by an increase in dosage of any of the concomitant medications and defined by mucosal healing on endoscopic examination (score of zero on Geboes scaled).

### Example 32

This example describes an exemplary protocol by administering pluripotent stem cells (or progeny) intrathecally.

Intrathecal administration is performed using a protocol similar to the one described below. The patient will have to be properly interviewed; it is of special interest if the patient is anticoagulated for any reason. The procedure is explained to the patient and any questions answered. The informed consent forms and other paper work are competed.
1. The patient arrives to the area were the procedure is going to take place.
2. Hospital gown is given to the patient and he is instructed to change into it, with the opening in the back.
3. The patient is instructed to lie down on his back on the bed.
4. Nursing staff places an IV access (this is standard safety procedure, in case of a complication presenting itself and fast IV access is required).
5. The specialist, who is going to perform the procedure, instructs the patient to position himself in the way that the physician considers optimal to open the intervertebral space.
6. The patient's lower back area is cleaned with a solution of alcohol and iodine and sterile drapes are placed.
7. The specialist injects local anesthesia to numb the injection area.
8. The specialist then uses the intrathecal injection needle and inserts it into the intervertebral space of either L3 and L4 or L4 and L5.
9. The specialist waits to see clear cerebrospinal fluid come out of the end of the intrathecal injection needle, to be sure that he is in the right space.
10. The physician then injects the stem cell preparation slowly.
11. After finishing the injection, the intrathecal needle is taken out.
12. The nursing staff then cleans the patient's lower back from the alcohol and iodine solution and places a band-aid on the injection site.
13. The patient is instructed to lie flat on his back on the bed.
14. The patient is observed for 20 minutes to make sure that no adverse reactions are seen.
15. The patient's IV is then discontinued and the patient is instructed to change into his clothes.
16. The patient is then discharged and told to lie flat on his back for about 6 hours when returning home to minimize the risk of headaches, which is the most common side effect.

### Example 33

This example describes manufacture and quantification of pluripotent stem cell-conditioned media (CM).

CM is generated as described in Example 10. In order to quantitate biological activity, dilutions of CM in the following ratios by volume 1:1, 1:10, 1:100, 1:1000, are made in DMEM in absence of fetal calf serum or other serum sources, and said diluted media is added to a 200 uL culture of 5×10³ human cord blood isolated CD34+ cells per well in 96 well plates in a 48 hour culture condition. Depending on biological need (e.g., angiogenesis, protection from apoptosis, etc.) other biological outputs may be used. The proliferation of the CD34 cells is quantitated by a tritiated thymidine method. Briefly, 1 μCi of [³H]thymidine (Amersham) was added to each well for the last 12 h of culture. At the end of the culture period, using an automated cell harvester, the cells are collected onto glass microfiber filter, and the radioactive labeling incorporation was measured by a Wallac Betaplate liquid scintillation counter. 1 Unit of CM activity was designated as the amount of CM needed to stimulate proliferation of cord blood derived CD34+ cells by 100% higher than said cells in DMEM alone. Calculations are made on a logarithmic curve as described for other biological agents whose activity is quantitated in Units, DeKoter, et. al., Cell Immunol. 175:120 (1997).

In order to concentrate CM, a volume of 4 litres of media is lyophilized under sterile conditions. Lyophilate was subsequently dialyzed using an exclusion of 5000 Daltons in order to extract salts and other small molecules in the solution. Reconstitution was performed in various volumes of USP saline and sterility as well as activity was quantified. Based on activity as measured using the CD34+ stimulation assay, various batches of ERCCM were manufactured which are used for some of the experiments described below.

### Example 34

This example describes a protocol for using pluripotent stem cell-conditioned media (CM) to treat an animal stroke model.

C57BL/6 (Jackson Laboratory) mice weighing approximately 25 grams each are given free access to food and water before and during the study. Animals are acclimated to the laboratory environment for 1 week prior to experimentation. Four groups of 10 mice each are treated by intravenouos infusion as follows: Group 1 vehicle, Group 2 FGF-1 (10 mg/kg), Group 3 ERCCM (100 U/kg) and Group 4 FGF-1 together with CM. Mice are infused intravenously, 1 hour after the initiation of ischemia. CM is generated, concentrated, and units of activity are quantified as previously described.

Each mouse is subjected to one hour of cerebral ischemia followed by 24 hours of reperfusion. At the end of the ischemic period, animals are treated as described above and at 14 days are examined for infarct volume. Each mouse is anesthetized and a thermistor probe is inserted into the rectum to monitor body temperature, which is maintained at 36-37° C. by external warming. The left common carotid artery (CCA) is exposed through a midline incision in the neck. The superior thyroid and occipital arteries are electrocoagulated and divided. A microsurgical clip is placed around the origin of the internal carotid artery (ICA). The distal end of the ECA is ligated with 6-0 silk and transected. A 6-0 silk is tied loosely around the ECA stump. The clip is removed and the fire-polished tip of a 5-0 nylon suture (poly-L-lysine coated) is gently inserted into the ECA stump. The loop of the 6-0 silk is tightened around the stump and the nylon suture is advanced approximately 11 mm (adjusted for body weight) into and through the internal carotid artery (ICA) after removal of the aneurysm clip, until it rests in the anterior cerebral artery (ACA), thereby occluding the anterior communicating and middle cerebral arteries. The animal is returned to home cage after removal from anesthesia. After the nylon suture is been in place for 1 hour, the animal is re-anesthetized, rectal temperature is recorded, the suture is removed and the incision closed.

Neurological deficits are assessed 14 days after ischemia based on a scale from 0 (no deficits) to 4 (severe deficits) as commonly used in the discipline. Neurological scores are as follows: 0, normal motor function; 1, flexion of torso and contralateral forelimb when animal is lifted by the tail; 2, circling to the contralateral side when held by the tail on a flat surface, but normal posture at rest; 3, leaning to the contralateral side at rest; 4, no spontaneous activity. For infarct volume determination after behavioral testing, the animals are anesthetized with an intraperitoneal injection of sodium pentobarbital (50 mg/kg). The brains are removed, sectioned into 4 2-mm sections through the infracted region and placed in 2% triphenyltetrazolium chloride (TTC) for 30 minutes at 24 hours. Subsequently, the sections are placed in 4% paraformaldehyde over night. The infarct area in each section is determined with a computer-assisted image analysis system, consisting of a computer equipped with a Quick Capture frame grabber card, Hitachi CCD camera mounted on a camera stand. NIH Image Analysis Software, v. 1.55 is used for quantification of image data.

The images are captured and the total area of infarct is determined over the sections. A single operator blinded to treatment status performs all measurements. Summing the infarct volumes of the sections calculated the total infarct volume.

1. A human pluripotent stem cell that expresses a marker selected from CD29, CD41a, CD44, CD90, and CD105, and having an ability to proliferate at a rate of 0.5-1.5 doublings per 24 hours in a growth medium.

2. The human pluripotent stem cell of claim 1, wherein said cell further expresses a marker selected from NeuN, CD9, CD62, CD59, Actin, GFAP, NSE, Nestin, CD73, SSEA-4, hTERT, Oct-4, and tubulin.

3. The human pluripotent stem cell of claim 1, wherein said cell further expresses a marker selected from hTERT and Oct-4, but does not express a STRO-1 marker, and has an ability to undergo cell division in less than 24 hours in a growth medium.

4. The human pluripotent stem cell of claim 1, wherein said cell further expresses a STRO-1 marker, and has an ability to proliferate at a rate of 0.5-0.9 doublings per 24 hours in a growth medium.

5. (canceled)

6. The human pluripotent stem cell of claim 1, wherein said cell produces matrix metalloprotease 3 (MMP3), matrix metalloprotease 10 (MMP10), GM-CSF, PDGF-BB or angiogenic factor ANG-2.

7. The human pluripotent stem cell of claim 1, wherein said cell is derived or originates from endometrium, endometrial stroma, endometrial membrane, or menstrual blood.

8.-18. (canceled)

19. The human pluripotent stem cell of claim 1, wherein said cell is capable of differentiating into an adipogenic, endothelial, hepatic, osteogenic, neural, pancreatic or myocytic cell lineage.

20. The human pluripotent stem cell of claim 1, wherein said cell has a stable karyotype for at least 10, 20, 30, 40, 50, 60, 70, 80, 90, 100 or more cell divisions.

21.-23. (canceled)

24. The cell population of claim 1, wherein said cell is capable of stimulating, inducing, increasing, promoting, enhancing or augmenting a reparative process in a host.

25. The human pluripotent stem cell of claim 1, wherein said cell is capable of suppressing, inhibiting, reducing, decreasing, preventing, blocking, limiting or controlling a T cell mediated response in vitro or in vivo.

26.-27. (canceled)

28. The human pluripotent stem cell of claim 1, wherein said mesenchymal cell marker is one or more of: CD54, CD106, an HLA-I marker, vimentin, ASMA, collagen-1, or fibronectin, but not a HLA-DR, CD1 17, or a hemopoietic cell marker.

29.-42. (canceled)

43. The human pluripotent stem cell of claim 1 wherein said cell or cells is capable of stimulating angiogenesis, inhibiting fibrosis or scar tissue formation, inhibiting inflammation, inhibiting undesired or pathological apoptosis.

44. The human pluripotent stem cell of claim 1 wherein said cell or cells is capable of stimulating endogenous progenitor cell proliferation, stimulation of endogenous stem cell proliferation, stimulation of endogenous progenitor cell differentiation, stimulation of endogenous stem cell differentiation, stimulation of exogenous progenitor cell proliferation, stimulation of exogenous stem cell proliferation, stimulation of exogenous progenitor cell differentiation, stimulation of exogenous stem cell differentiation.

45.-56. (canceled)

57. A medium incubated with the cell population of claim 1 for a period of about 1-72 hours, 3-7 days, or more.

58. The medium of claim 57, wherein the medium comprises a matrix metalloprotease 3 (MMP3), matrix metalloprotease 10 (MMP10), GM-CSF, PDGF-BB or angiogenic factor ANG-2.

59. The medium of claim 57, wherein said medium stimulates, increases, induces, enhances or augments cell survival, viability, growth, proliferation or differentiation of a totipotent stem cell, a pluripotent stem cell, a multipotent stem cell or a differentiated cell.

60. (canceled)

61. A method of producing a conditioned growth medium comprising, contacting a liquid growth medium with the cell population of claim 1 under conditions suitable for viability of said population of cells for a period of about 1-72 hours, 3-7 days or more.

62.-64. (canceled)

65. A method of stimulating hematopoiesis comprising administering to a subject the conditioned growth medium of claim 57 in an amount sufficient to stimulate hematopoiesis.

66. A method of inhibiting inflammation comprising administering to a subject the conditioned growth medium of claim 57 in an amount sufficient to inhibit inflammation.

67. A method of treating a subject in need of stimulation of angiogenesis, comprising administering the cell population of claim 1 to the subject in an amount sufficient to treat the subject.

68.-76. (canceled)

77. A method of treating a subject in need of osteocytes or an osteocyte function comprising administering the cell population of claim 1 to the subject in an amount sufficient to increase osteocyte numbers, stimulate osteocyte formation or increase or stimulate an osteocyte function.

78.-80. (canceled)

81. A method of treating a subject having or at risk of having a neurological or muscular disease or disorder, comprising administering the cell population of claim 1 to the subject in an amount sufficient to treat the neurological or muscular disease or disorder.

82.-112. (canceled)

113. A method of increasing numbers of T regulatory cells in a subject, comprising administering human pluripotent stem cell of claim 1, or the population or plurality of cells of claim 1, to a subject under conditions facilitating increased numbers of T regulatory cells, thereby increasing numbers of T regulatory cells in the subject.

114.-127. (canceled)

128. A method of stimulating, increasing, inducing, augmenting, or enhancing immunological tolerance or treating an autoimmune disorder comprising administering the cell population of claim 1 to the subject in an amount sufficient to stimulate, increase, induce, augment, or enhance immunological tolerance or treat the autoimmune disorder.

129. (canceled)

130. A method of stimulating, increasing, inducing, augmenting, or enhancing hematopoiesis comprising administering the cell population of claim 1 to the subject in an amount sufficient to stimulate, increase, induce, augment or enhance hematopoiesis.

131. A method of stimulating, increasing, inducing, augmenting, or enhancing Angiogenesis comprising administering the cell population of claim 1 to the subject in an amount sufficient to stimulate, increase, induce, augment or enhance angiogenesis.
